(19) United States
(12) Patent Application Publication
(10) Pub. No.: US 2024/0052420 A1
(43) Pub. Date: Feb. 15, 2024

(54) TREATMENT RESPONSE PREDICTIVE METHOD

(71) Applicants: The Institute of Cancer Research: Royal Cancer Hospital, London (GB); Cancer Research Technology Limited, London (GB); Breast Cancer Now, London (GB)

(72) Inventors: Mitchell Dowsett, London (GB); Eugene Francis Schuster, London (GB); Chon U Maggie Cheang, London (GB)

(73) Assignees: The Institute of Cancer Research: Royal Cancer Hospital, London (GB); Cancer Research Technology Limited, London (GB); Breast Cancer Now, London (GB)

(21) Appl. No.: 18/246,643

(22) PCT Filed: Sep. 24, 2021
(86) PCT No.: PCT/EP2021/076368

§ 371 Date: Mar. 24, 2023

(30) Foreign Application Priority Data
Sep. 25, 2020 (GB) 2015200.5

(51) Int. Cl. C12Q 1/6886; G16B 25/10; G16B 40/20; G16H 50/20; A61K 31/4196

(52) U.S. Cl. CPC C12Q 1/6886; G16B 25/10; G16B 40/20; G16H 50/20; A61K 31/4196; C12Q 2600/106; C12Q 2600/158

(57) ABSTRACT

The present invention provides a method for predicting whether a human subject having breast cancer will be resistant to, or sensitive to, therapy with a cyclin-dependent kinase (CDK) inhibitor, the method comprising: a) measuring the gene expression in a sample obtained from the breast tumour of the patient to obtain a sample gene expression profile of at least the following modules: (i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KRT14, KNTC2, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T; (ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SFRS10, SFRS7, SNRPD1, STMN1 and TMPO; (iii) an RB1 module comprising the gene RB1; and (iv) a CCNE1 module comprising the gene CCNE1; and b) making a prediction of whether the subject will be resistant to or sensitive to said CDK inhibitor treatment based on the sample gene expression profile comprising said modules (i) to (iv). Also provided are related methods of treatment, computer-implemented methods of predicting treatment response and systems for use in such methods.

This application claims priority from GB2015200.5 filed 25 Sep. 2020, the contents and elements of which are herein incorporated by reference for all purposes.

### FIELD OF THE INVENTION

The present invention relates to materials and methods for predicting response to cyclin-dependent kinase (CDK) inhibitors, particularly CDK4/6 inhibitors, among cancer patients, particularly patients having breast cancer who are undergoing or will be treated with endocrine therapy, such as with an aromatase inhibitor.

### BACKGROUND TO THE INVENTION

The majority of early breast cancer tumours in postmenopausal women are ER+and HER2−. Treatment is usually surgery, followed by chemotherapy/radiotherapy as indicated, and endocrine therapy for all patients. In postmenopausal women the most effective endocrine therapy agents are aromatase inhibitors (AIs). However, many patients recur because of de novo or acquired resistance to AI: approximately two thirds of women who die from breast cancer will have initially presented with ER+/HER2− disease. This amounts to 8000 deaths/year in the UK.

In the setting of advanced breast cancer CDK4/6 inhibitors including palbociclib, abemaciclib and ribociclib, have been found to be highly effective agents when combined with endocrine therapy in extending progression free and survival. Abemaciclib alone may also be used to treat these cancers to treat ER+/HER2− cancers that have progressed during past hormone therapy and chemotherapy. A small number of studies have examined the combination of an AI with a CDK4/6 inhibitors presurgical (neoadjuvant) treatment for ER+/HER2− disease. Clinical studies are underway to determine the effectiveness of CDK4/6 inhibitor when combined with an AI (or other endocrine therapy) in comparison with endocrine therapy alone in the adjuvant treatment of primary breast cancer.

These adjuvant studies are being conducted in broad hormone sensitive early breast cancer patient populations where higher risk of recurrence is predicted largely based on clinical risk factors—tumour size and nodal involvement and are expected to report in about 2 years' time. A robust biomarker signature to identify subgroups of patients who are likely to derive most benefit from adding CDK4/6 inhibitors to endocrine therapy will become a high priority for breast cancer clinical management at that time.

The AIR-CIS (Aromatase Inhibitor Resistant-CDK4/6 Inhibitor Sensitive) algorithm devised by the present inventors will characterise the subgroup of patients receiving AI therapy who are most likely to gain relative benefit from addition of CDK4/6 inhibitors.

### BRIEF DESCRIPTION OF THE INVENTION

The present inventors have devised an algorithm, Aromatase Inhibitor Resistant-CDK4/6 Inhibitor Sensitive (AIR-CIS), that classifies the subgroup of patients receiving AI therapy who are most likely to gain relative benefit from addition of CDK4/6 inhibitors.

Accordingly, in a first aspect the present invention provides a method for predicting whether a human subject having breast cancer will be resistant to, or sensitive to, therapy with a cyclin-dependent kinase (CDK) inhibitor, the method comprising:
a) measuring the gene expression in a sample obtained from the the patient to obtain a sample gene expression profile of the breast tumour of at least the following modules:
(i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1 (alias NUF2), CDH3, CENPF, CEP55, CXXC5, EGFR, ERBB2, ESR1, EXO1, FGFR4, FOXA1, FOXC1, GPR160, GRB7, KIF2C, KNTC2 (alias NDC80), KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SFRS10, SFRS7, SNRPD1, STMN1 and TMPO;
(iii) an RB1 module comprising the gene RB1; and
(iv) a CCNE1 module comprising the gene CCNE1; and
b) making a prediction of whether the subject will be resistant to or sensitive to said CDK inhibitor treatment based on the sample gene expression profile comprising said modules (i) to (iv).

In some embodiments the luminal vs. non-luminal module comprises the genes: ANLN, ESR1, PGR and SLC39A6.

In some embodiments the luminal vs. non-luminal module comprises the genes: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T.

In some embodiments the E2F module comprises the genes: SFRS1, DNAJC9, FBXO5, DCK, and TMPO.

In some embodiments the E2F module comprises the genes: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SFRS10, SFRS7, SNRPD1, STMN1 and TMPO.

In some embodiments the method further comprises measuring the gene expression in the sample of one or more housekeeping genes. The housekeeping genes comprise at least 2, 3, 4, 5, 6, 7, or at least 8 housekeeping genes selected from the group consisting of: ACTB, MRPL19, PSMC4, RPLP0, SF3A1, GUSB (alias GUS), PUM1 and TFRC.

In some embodiments the subject is predicted to be resistant to said CDK inhibitor therapy when at least one of the following is true:
(i) the luminal vs. non-luminal module classifies the sample as non-luminal;
(ii) the E2F module classifies the sample as having high E2F expression;
(iii) the RB1 module classifies the sample as having low RB1 expression; and
(iv) the CCNE1 module classifies the sample as having high CCNE1 expression,
and wherein the subject is predicted to be sensitive to said CDK inhibitor therapy otherwise.

In some embodiments,
(i) the luminal vs. non-luminal module classifies the sample as luminal;
(ii) the E2F module classifies the sample as having low E2F expression;
(iii) the RB1 module classifies the sample as having high RB1 expression; and
(iv) the CCNE1 module classifies the sample as having low CCNE1 expression,
and therefore the subject is predicted to be sensitive to said CDK inhibitor therapy.

In some embodiments the E2F module classifies a sample as having high E2F expression when the average log₂ gene expression of the E2F signature genes is greater than or equal to 9.392 or is greater than or equal to 9.446.

In some embodiments the RB1 module classifies the sample as having low RB1 gene expression when the log₂ gene expression measures less than or equal to 8.4068 or measures less than or equal to 8.4332.

In some embodiments the CCN1E module classifies the sample as having high CCN1E expression when the log e gene expression measures greater than or equal to 8.264 or measures greater than or equal to 7.9596.

In some embodiments the luminal vs. non-luminal module classifies the sample as luminal or non-luminal on the basis of the nearest centroid, wherein the sample gene expression profile of the genes of said luminal vs. non-luminal module is compared with reference centroids derived from measured gene expression of the said genes from a plurality of samples known to be of luminal phenotype and a plurality of samples known to be of non-luminal phenotype, respectively. The luminal vs. non-luminal classification may be made according to the PAM50 nearest centroid as disclosed in Parker et al., J Clin Oncol, 2009; 27(8):1160-1167, doi: 10.1200/JCO.2008.18.1370 (reference (3)).

In some embodiments the genes of the luminal vs. non-luminal module and corresponding reference centroids are selected from the following a) to f):

| Genes | Luminal | Non-Luminal |
|---|---|---|
| a) | | |
| ANLN | −0.436597966 | −0.171060579 |
| ESR1 | 0.724924673 | −0.293827849 |
| PGR | −0.14811616 | −0.58065462 |
| SLC39A6 | 2.236554031 | 1.642473045 |
| b) | | |
| ANLN | −0.436597966 | −0.171060579 |
| BCL2 | 0.214783191 | −0.257430908 |
| ESR1 | 0.724924673 | −0.293827849 |
| PGR | 0.243228161 | −0.14811616 |
| PHGDH | −0.008631878 | 0.414898718 |
| SLC39A6 | 2.236554031 | 1.642473045 |
| c) | | |
| ANLN | −0.436597966 | −0.171060579 |
| BCL2 | 0.214783191 | −0.257430908 |
| CENPF | 0.261621563 | 0.531497672 |
| ESR1 | 0.724924673 | −0.293827849 |
| PGR | 0.243228161 | −0.14811616 |
| PHGDH | −0.391826227 | −0.008631878 |
| RRM2 | 0.59104305 | 0.842673949 |
| SLC39A6 | 2.236554031 | 1.642473045 |
| d) | | |
| ANLN | −0.436597966 | −0.171060579 |
| BCL2 | 0.214783191 | −0.257430908 |
| CDC20 | −0.347286987 | −0.09561991 |
| CDH3 | −0.522006879 | −0.173025486 |
| CENPF | 0.261621563 | 0.531497672 |
| ESR1 | 0.724924673 | −0.293827849 |
| PGR | 0.243228161 | −0.14811616 |
| PHGDH | −0.391826227 | −0.008631878 |
| RRM2 | 0.363376998 | 0.59104305 |
| SLC39A6 | 2.236554031 | 1.642473045 |
| e) | | |
| ANLN | −0.436597966 | −0.171060579 |
| BCL2 | 0.214783191 | −0.257430908 |
| CDC20 | −0.347286987 | −0.09561991 |
| CDH3 | −0.522006879 | −0.173025486 |
| CENPF | 0.017447942 | 0.261621563 |
| ESR1 | 0.724924673 | −0.293827849 |
| PGR | 0.243228161 | −0.14811616 |
| PHGDH | −0.391826227 | −0.008631878 |
| PTTG1 | 0.028806637 | 0.23337021 |
| RRM2 | 0.363376998 | 0.59104305 |
| SLC39A6 | 2.236554031 | 1.642473045 |
| UBE2C | 0.060331049 | 0.318978765 |
| f) | | |
| ANLN | −0.436597966 | −0.171060579 |
| BCL2 | 0.214783191 | −0.257430908 |
| CDC20 | −0.574985771 | −0.347286987 |
| CDH3 | −0.522006879 | −0.173025486 |
| CENPF | 0.017447942 | 0.261621563 |
| ESR1 | 0.724924673 | −0.293827849 |
| FOXA1 | 0.774898643 | 0.173550394 |
| MLPH | 0.825757051 | 0.357073665 |
| PGR | 0.243228161 | −0.14811616 |
| PHGDH | −0.391826227 | −0.008631878 |
| PTTG1 | 0.028806637 | 0.23337021 |
| RRM2 | 0.363376998 | 0.59104305 |
| SLC39A6 | 2.236554031 | 1.642473045 |
| UBE2C | 0.060331049 | 0.318978765 |

In some embodiments the gene expression level of one or more of said genes is measured using NanoString nCounter Analysis. In some embodiments the gene expression level of said genes is measured by measuring tumour derived RNA in a biological sample, e.g. a plasma or blood sample. Such non-invasive techniques may be preferred in certain clinical situations.

In some embodiments the gene expression level of one or more of said genes may be measured using a technique other than NanoString (e.g. RT-PCR) and then adjusted to NanoString equivalent values by applying gene-wise linear conversion factors. The linear conversion factors (e.g. slope and intercept) for each gene may be derived as described in detail herein. In particular embodiments the gene-wise linear conversion factor for each gene may be determined by linear regression analysis of gene expression measurements made of the same sample by NanoString and the alternative measurement method (e.g. RT-PCR).

In some embodiments the gene expression measurements are normalised by reference to the expression of one or more housekeeping genes. Housekeeping genes are determined by selecting genes that minimize the pairwise variation statistic from a large dataset of ER+ postmenopausal patients.

In some embodiments the subject has ER+ and HER2− breast cancer. In some embodiments the subject is female, e.g. a postmenopausal woman.

In some cases the subject has been treated with, is undergoing treatment with, or is planned to have treatment with, endocrine therapy, particularly treatment with an aromatase inhibitor (e.g. anastrozole or letrozole).

In some embodiments the sample has been obtained from the subject at least one week or at least two weeks after the subject commenced treatment with an aromatase inhibitor (e.g. anastrozole and letrozole).

In some embodiments the subject has had surgical removal of a breast tumour.

In some embodiments the CDK inhibitor therapy comprises treatment with a CDK4/6 inhibitor, such as palbociclib, abemaciclib or ribociclib.

In some cases the breast tumour of the subject exhibits a marker of proliferation Ki-67 (MKI67) score of 8% or greater, meaning 8% or more tumour cells are positive for Ki-67 expression. As used herein Ki67_{B} means the Ki67 measurement at baseline; Ki67_{2wk} means the Ki67 measurement after 2 weeks of aromatase inhibitor treatment.

In some embodiments the subject is predicted to be sensitive to said CDK inhibitor therapy, and wherein the method further comprises the step of administering, or recommending administration of, a therapeutically effective amount of a CDK inhibitor, optionally a CDK4/6 inhibitor, such as such as palbociclib, abemaciclib or ribociclib.

In some embodiments the CDK inhibitor is administered as part of a combination therapy with endocrine therapy, such as an aromatase inhibitor.

In some embodiments the method comprises concurrent, sequential or separate administration of:
(i) palbociclib, abemaciclib or ribociclib; and
(ii) anastrozole or letrozole,
to the subject in therapeutically effective amounts.

In some embodiments the subject is predicted to be resistant to said CDK inhibitor therapy, and wherein the method further comprises administering endocrine therapy (e.g. an aromatase inhibitor) to the subject in the absence of any CDK4/6 inhibitor therapy. In this way subjects who are unlikely to benefit from the addition of CDK4/6 inhibitor therapy may be spared such therapy and any related unwanted side effects.

In a second aspect the present invention provides a computer-implemented method for predicting whether a human subject having breast cancer will be resistant to, or sensitive to, therapy with a cyclin-dependent kinase (CDK) inhibitor, the method comprising:
a) obtaining gene expression data representing the gene expression profile of a sample obtained from the breast tumour of the subject of at least the following modules:
(i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) an RB1 module comprising the gene RB1; and
(iv) a CCNE1 module comprising the gene CCNE1; and
b) comparing the gene expression data obtained in a) with reference gene expression profiles for each of said modules, optionally wherein the gene expression data and the reference gene expression profiles comprise gene expression measurements that have been normalised to one or more housekeeping genes;
c) classifying the subject as resistant to said CDK inhibitor therapy if at least one of the following is true:
(i) the luminal vs. non-luminal module classifies the sample as non-luminal;
(ii) the E2F module classifies the sample as having high E2F expression;
(iii) the RB1 module classifies the sample as having low RB1 expression; and
(iv) the CCNE1 module classifies the sample as having high CCNE1 expression,
or classifying the subject as sensitive to said CDK inhibitor therapy otherwise.

In a third aspect the present invention provides a system for predicting treatment response of a human subject having breast cancer to therapy with a cyclin-dependent kinase (CDK) inhibitor, the system comprising:
A) a plurality of oligonucleotide probes for detection of gene transcripts of the following genes:
(i) at least four genes from the luminal vs. non-luminal module consisting of the genes: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) at least five genes from the E2F module consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) the RB1 gene; and
(iv) the CCNE1 gene;
   B) a computer having at least one processor and at least one non-transitory computer readable medium containing instructions that, when executed by the at least one processor, cause the at least one processor to perform operations comprising:
   (a) receiving gene expression data representing the gene expression profile of a sample obtained from the breast tumour of a human subject having breast cancer of at least the following modules:
(i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) an RB1 module comprising the gene RB1; and
(iv) a CCNE1 module comprising the gene CCNE1; and
(b) comparing the gene expression data with reference gene expression profiles for each of said modules, optionally wherein the gene expression data and the reference gene expression profiles comprise gene expression measurements that have been normalised to one or more housekeeping genes; and
(c) classifying the subject as resistant to said CDK inhibitor therapy if at least one of the following is true:
(i) the luminal vs. non-luminal module classifies the sample as non-luminal;
(ii) the E2F module classifies the sample as having high E2F expression;
(iii) the RB1 module classifies the sample as having low RB1 expression; and
(iv) the CCNE1 module classifies the sample as having high CCNE1 expression,
or classifying the subject as sensitive to said CDK inhibitor therapy otherwise.

In some embodiments the plurality of probes comprise NanoString nCounter probes.

In some embodiments the system of the third aspect of the invention may be for use in the method of the first aspect of the invention.

In a fourth aspect the present invention provides a CDK4/6 inhibitor for use in a method of treatment of breast cancer in a human subject, wherein the method of treatment comprises carrying out the method of the first aspect of the invention on a sample obtained from the subject whereupon the subject is predicted to be sensitive to the CDK4/6 inhibitor (e.g. palbociclib, abemaciclib or ribociclib). Patients identified as likely to benefit from the addition of CDK4/6 inhibitor therapy constitute a novel patient subpopulation who can be expected to derive greatest benefit from such treatment.

In some embodiments the treatment further comprises concurrent, sequential or separate administration of endocrine therapy (e.g. with an aromatase inhibitor such as anastrozole or letrozole).

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1—Effect of BC subtype on sensitivity to CDK4/6 inhibitor.

FIG. 2—Response to palbociclib is governed by BC sub-type (taken from Finn et al SABCS 2017)

FIG. 3—Assessment of E2F signatures in data from NeoPalAna

FIG. 4—High CCNE1 expression is associated with poor response to CDK4/6 inhibition in metastatic tissue (taken from Turner et al AACR 2018)

FIG. 5—Expression of CCNE1 associates with resistance to palbociclib in NeoPalAna (taken from MA et al 2017)

FIG. 6—Shows a Venn diagram of the number of patients in each module among the 25 AI-treated patients in POETIC that would be eligible for randomisation in POETIC-A (i.e. with Ki67B≥20% and on-treatment Ki672w≥8%) and predicted to be resistant to abemaciclib

FIG. 7—Venn diagram of 115 tumours with 53 tumours considered as sensitive and 62 as resistant

FIG. 8—Log-2 RB1 expression by Nanostring in 52 samples from POETIC taken after 2 weeks' AI and with baseline and 2 week Ki67>20% and >8%, respectively

FIG. 9—Classification error for Luminal vs non-Luminal cases using different gene lists

FIG. 10—Venn diagram showing the break down in terms of AI-resistant tumours with overlapping CDK4/6 resistance modules as defined in AIR-CIS. As shown, 6 samples showed four resistant modules, 8 showed three resistant modules, 9 showed 2 resistant modules, 17 showed 1 resistant module and 40 showed as CDK4/6 sensitive.

### DETAILED DESCRIPTION OF THE INVENTION

In describing the present invention, the following terms will be employed, and are intended to be defined as indicated below.

### Samples

A “test sample” as used herein may be a cell or tissue sample (e.g. a biopsy), a biological fluid, an extract (e.g. a protein or DNA extract obtained from the subject). In particular, the sample may be a tumour sample, including a breast tumour (primary or secondary). The sample will generally be comprise nucleic acid (e.g. RNA or DNA) and/or protein. In some cases the sample may be a blood or plasma sample containing tumour-derived RNA. Measurement of gene expression may involving quantification of RNA from a sample, including a blood or plasma sample. The sample may be one which has been freshly obtained from the subject or may be one which has been processed and/or stored prior to making a determination (e.g. frozen, fixed or subjected to one or more purification, enrichment or extractions steps). In embodiments, the sample is a fixed tumour tissue sample (such as e.g. a formalin-fixed paraffin-embedded (FFPE) tissue sample), or a frozen tumour tissue sample (such as e.g. a fresh frozen (FF) tissue sample). The preferred sample type according to the present invention is a FFPE tissue sample, as this type of samples is widely available. Indeed, FFPE tissue samples are commonly obtained in clinical settings, for example for histopathological diagnosis. Reference to “cancer cells” herein may refer to cancer cells present in a cell or tissue sample, such as e.g. cells in a tumour tissue from a biopsy.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Gene Expression

Reference to determining the expression level refers to determination of the expression level of an expression product of the gene. Expression level may be determined at the nucleic acid level or the protein level. Within the context of the present invention, expression levels of genes of interest are preferably determined at the nucleic acid level, and in particular at the mRNA level.

The gene expression levels determined may be considered to provide an expression profile. By “expression profile” is meant a set of data relating to the level of expression of one or more of the relevant genes in an individual, in a form which allows comparison with comparable expression profiles (e.g. from individuals for whom the prognosis is already known), in order to assist in the determination of prognosis and in the selection of suitable treatment for the individual patient.

The determination of gene expression levels may involve determining the presence or amount of mRNA in a sample of cancer cells or a sample containing material derived from cancer cells (e.g. a blood, plasma, urine or other biological liquid comprising tumour-derived nucleic acids, such as circulating tumour RNA). Gene expression levels may be determined in a sample of cancer cells using any conventional method, for example using nucleic acid microarrays or using nucleic acid synthesis (such as quantitative PCR). For example, gene expression levels may be determined using a NanoString nCounter Analysis system (see, e.g., U.S. Pat. No. 7,473,767). In some cases, a blood sample may be analysed to measure tumour derived RNA in order to quantify gene expression of the genes of the modules of the present invention (see, e.g., Xue et al., 2019, Nature Scientific Reports (2019) 9:12943|https://doi.org/10.1038/s41598-019-49445-x, describing measurement of tumour gene expression by RNA sequencing of patient blood or plasma samples). As described herein (see, e.g., Example 7), the present inventors found that the AIR-CIS classification based on RNA-seq data showed remarkable concordance with that based on gene expression data determined with Nanostring methodology. This shows that the method and system of the invention is not particularly limited as regards the technique used to measure gene expression.

Importantly, the order in which different genes making up the AIR-CIS panel, individual modules thereof and/or genes within a given module are analysed to determine gene expression is not particularly limited. It is possible that gene expression for all genes of interest may be determined from a sample in parallel such as in a single assay or as multiple assays on the same day. However, it is specifically contemplated that the gene expression of any given gene may be determined separately from determination of one or more other genes. In particular, gene expression of the gene or genes making up a particular module as defined herein may be determined separately from the gene or genes of other modules, such as being determined on different days, by different labs, and/or using different techniques.

Gene expression measurements in accordance with the method of the present invention (e.g. one or more AIR-CIS modules) may be combined with other known predictive gene signatures, such as those having clinical relevance. In one particular embodiment contemplated herein, one or more (such as all) of the AIR-CIS modules as defined herein may be combined with the PAM50 gene expression signature (see, e.g., reference (3) incorporated herein by reference).

Alternatively or additionally, the determination of gene expression levels may involve determining the protein levels expressed from the genes in a sample containing cancer cells obtained from an individual. Protein expression levels may be determined by any available means, including using immunological assays. For example, expression levels may be determined by immunohistochemistry (IHC), Western blotting, ELISA, immunoelectrophoresis, immunoprecipitation and immunostaining. Using any of these methods it is possible to determine the relative expression levels of the proteins expressed from the genes listed in Table 1.

Gene expression levels may be compared with the expression levels of the same genes in cancers from a group of patients whose survival time and/or treatment response is known. The patients to which the comparison is made may be referred to as the ‘control group’. Accordingly, the determined gene expression levels may be compared to the expression levels in a control group of individuals having cancer. The comparison may be made to expression levels determined in cancer cells of the control group. The comparison may be made to expression levels determined in samples of cancer cells from the control group. The cancer in the control group may be the same type of cancer as in the individual. For example, if the expression is being determined for an individual with breast cancer, the expression levels may be compared to the expression levels in the cancer cells of patients also having breast cancer.

Other factors may also be matched between the control group and the individual and cancer being tested. For example the stage of cancer may be the same, the subject and control group may be age-matched and/or gender matched.

Additionally the control group may have been treated with the same form of surgery and/or same therapeutic agent(s).

Accordingly, an individual may be stratified or grouped according to their similarity of gene expression with the group previously identified as resistant to or sensitive to CDK4/6 inhibitor therapy.

### Methods for Classification Based on Gene Expression

In some embodiments, the present invention provides methods for classifying or monitoring breast cancer in subjects. In particular, data obtained from analysis of gene expression may be evaluated using one or more pattern recognition algorithms. Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from samples of known subgroup (e.g., from subjects known to have a particular breast cancer CDK4/6 inhibitor response), and second to classify an unknown sample (e.g., “test sample”) according to subgroup (likely responder or likely non-responder).

Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyse data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm.

The other approach is termed “supervised” whereby a training set of samples with known class or outcome is used to produce a mathematical model which is then evaluated with independent validation data sets. Here, a “training set” of gene expression data is used to construct a statistical model that predicts correctly the “subgroup” of each sample. This training set is then tested with independent data (referred to as a test or validation set) to determine the robustness of the computer-based model. These models are sometimes termed “expert systems”, but may be based on a range of different mathematical procedures such as support vector machine, decision trees, k-nearest neighbour and naïve Bayes. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic gene expression profile. It is also possible to obtain confidence limits on any predictions, for example, a level of probability to be placed on the goodness of fit. The robustness of the predictive models can also be checked using cross-validation, by leaving out selected samples from the analysis.

After stratifying the training samples according to subtype, a centroid-based prediction algorithm may be used to construct centroids based on the expression profile of the gene sets described herein, e.g. the 81-gene AIR-CIS signature in Table 1 or a compact signature as described herein.

“Translation” of the descriptor coordinate axes can be useful. Examples of such translation include normalization and mean-centring. “Normalization” may be used to remove sample-to-sample variation. Some commonly used methods for calculating normalization factor include: (i) global normalization that uses all genes on the microarray or nanostring codeset; (ii) housekeeping genes normalization that uses constantly expressed housekeeping/invariant genes; and (iii) internal controls normalization that uses known amount of exogenous control genes added during hybridization (Quackenbush, 2002). In one embodiment, the genes forming the AIR-CIS signature can be normalized to one or more control housekeeping genes. Exemplary housekeeping genes include ACTB, MRPL19, PSMC4, RPLP0, SF3A1, GUSB (alias GUS), PUM1 and TFR. It will be understood by one of skill in the art that the methods disclosed herein are not bound by normalization to any particular housekeeping genes, and that any suitable housekeeping gene(s) known in the art can be used. Many normalization approaches are possible, and they can often be applied at any of several points in the analysis. In one embodiment, microarray data is normalized using the LOWESS method, which is a global locally weighted scatterplot smoothing normalization function. In another embodiment, qPCR and NanoString nCounter analysis data is normalized to the geometric mean of a set of multiple housekeeping genes. Moreover, qPCR can be analysed using the fold-change method.

“Mean-centering” may also be used to simplify interpretation for data visualisation and computation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centring and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centred and unit variance scaled. This technique is a very useful because each descriptor is then weighted equally, and large and small values are treated with equal emphasis. This can be important for genes expressed at very low, but still detectable, levels.

When comparing data from multiple analyses (e.g., comparing expression profiles for one or more test samples to the centroids constructed from samples collected and analysed in an independent study), it will be necessary to normalize data across these data sets. In one embodiment, Distance Weighted Discrimination (DWD) is used to combine these data sets together (Benito et al. (2004), incorporated by reference herein in its entirety). DWD is a multivariate analysis tool that is able to identify systematic biases present in separate data sets and then make a global adjustment to compensate for these biases; in essence, each separate data set is a multi-dimensional cloud of data points, and DWD takes two points clouds and shifts one such that it more optimally overlaps the other. Further methods for combining data sets include the “ComBat” method and others described in Lagani et al. 2016, the entire contents of which is expressly incorporated herein by reference. ComBat is a method specifically devised for removing batch effects in gene-expression data (Johnson W E, Li C, Rabinovic A. 2007, the entire contents of which is expressly incorporated herein by reference).

In some embodiments described herein, the prognostic performance of the gene expression signature and/or other clinical parameters is assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., gene expression profile with or without additional clinical factors, as described herein). The “hazard ratio” is the risk of death (or event such as a recurrence of the cancer) at any given time point for patients displaying particular prognostic variables.

### Genes Making Up the Gene Signature or Gene Expression Profile

In accordance with any aspect of the present invention, the genes that make up the gene expression profile (AIR-CIS signature) may be selected from those listed in Table 1, wherein the genes include all four of the modules: (i) luminal vs. non-luminal; (ii) RB1; (iii) E2F; and (iv) CCNE1. Particular subsets of the said genes are contemplated herein. For example, the “compact” 11-gene set: ANLN, ESR1, PGR, SLC39A6, SFRS1, DNAJC9, FBXO5, DCK, TMPO, RB1 and CCNE1.

### Breast Cancer

As used herein, “breast cancer” refers to any cancer of the breast, including, in particular, ER+ and HER2− primary breast cancer. A breast cancer patient may be undergoing or may be a candidate for surgery, medical therapy (including endocrine therapy, chemotherapy, CDK inhibitor therapy and/or monoclonal antibody therapy) and/or radiotherapy.

### Surgery

As used herein, “breast cancer surgery” or similar terms refer to physical removal of a breast tumour, optionally together with removal of surrounding tissue and/or lymph nodes. A breast cancer patient as contemplated herein may have had or may be a candidate for breast cancer surgery.

### Endocrine Therapy

As used herein, “endocrine therapy” or “hormonal therapy” includes therapy with agents intended to block hormone receptors (e.g. tamoxifen) or to block production of oestrogen such as an aromatise inhibitor (AI), e.g. anastrozole or letrozole.

### CDK Inhibitor

As used herein, “CDK inhibitor” includes CDK4/6 inhibitors such as palbociclib, abemaciclib and ribociclib. Moreover, agents that are being or will be developed to inhibit CDK, particularly CDK4 and CDK6, such as trilaciclib, are specifically contemplated herein.

The following is presented by way of example and is not to be construed as a limitation to the scope of the claims.

### EXAMPLES

### Materials and Methods

### Derivation of the AIR-CIS Algorithm Using Data from the POETIC Trial

The POETIC trial is a phase III, multicentre, randomised trial for postmenopausal women with ER/PR positive invasive breast cancer to determine whether 2 weeks perioperative aromatase inhibitor (AI) therapy before and after surgery improves outcome compared with standard adjuvant therapy alone. The trial organisers intend to perform translational work to determine the most effective time points for molecular profiling and measurement of proliferation marker Ki67 in order to predict long term outcome and time to recurrence, respectively. 4,486 patients were recruited from 130 UK sites over a 5.5 year period. Patients received either perioperative therapy with an AI for 4 weeks (two weeks before and two weeks after surgery) or no perioperative therapy. Patients will be followed up for at least 10 years.

NanoString nCounter was used to measure the gene expression of 81 genes (see Table 1) in RNA samples extracted from primary breast cancer samples. The phenotyping performed by the present inventors was assessed on tumours that had been treated with an AI for 2 weeks such that rewiring of the tumour that occurs over that time and is associated with continued proliferation could be captured.

Gene expression data were used to calculate values for four modules that together inform the predicted sensitivity or resistance to CDK4/6 inhibition. These modules include (i) intrinsic subtype classification, (ii) RB1 loss, (iii) E2F gene signature and (iv) CCNE1 expression.

### Measurement of the AIR-CIS in the POETIC-A Trial

The gene expression AIR-CIS profile is measured in tissue sections from the surgical core-cut biopsy of patients with Ki67≥8% after 2-weeks. A tumour being AIR-CIS negative (i.e. putative resistant) is defined as non-luminal subtype, and/or having expression for any of the 3 respective modules (E2F, CCNE1 and RB-loss) above predefined thresholds of expression.

100 ng of RNA is used on the nanostring platform. At least 2 core-cuts will be requested from the excision sample to minimise the likelihood of insufficient cells for Ki67 or RNA. For the RNA the present inventors' have shown that the AIR-CIS can be measured reliably on sections from the excision biopsy (unpublished data). The present inventors will therefore request this sample or sections from it if there is insufficient RNA from the core-cuts.

The 81-gene (including 8 house-keeper genes) code-set for measurement of the AIR-CIS will be identical throughout the study. Quality control samples will be included in each batch to ensure comparability and allow rejection if outside the bounds of acceptance. The values of the individual modules will be recorded such that in addition to the primary analysis of the benefit from added abemaciclib in relation to the AIR-CIS, a secondary analysis will be performed of benefit according to the individual 4 genes/modules in the AIR-CIS signature.

AIR-CIS analysis will be performed at the central laboratory at The Royal Marsden Hospital.

### Conversion Between Platforms for Measuring Gene Expression

Many different platforms exist for measuring gene expression levels. The examples provided herein use Nanostring nCounter gene expression data but it is expressly contemplated that the AIR-CIS algorithm can make use of data gathered using a different gene expression method or platform. For instance, data from a qPCR assay or Illumina sequencing can be converted to nanostring data before running the AIR-CIS algorithm. The inventors also envisage transferring this algorithm and expanding to other platforms such as RNA-seq data.

An exemplary method for translating data obtained by qPCR assay to nanostring data is provided herein.

### Computation and Verification of Conversion Factors for the Multi-Gene Prognostic Signature in a Training Set.

Gene-wise conversion factors for the multi-gene prognostic signature can be derived from a training cohort in which the gene expression levels are measured using both Nanostring and another measurement technique (e.g. RT-PCR). The present inventors have found that the gene expression measured by Nanostring and by RT-PCR for the genes employed in the gene signature of the present invention exhibit a linear relationship such that a linear regression model is able to provide reliable gene-wise conversion factors to convert between Nanostring and RT-PCR gene expression measurements or vice versa. In particular embodiments this can be achieved using linear regression to fit intercept and slope for the conversion and applying a cross-validation approach to select the conversion factors (intercept and slope) giving rise to the lowest error. In specific embodiments, the following strategy may be employed.

The conversion factors for each gene (intercept and slope) are estimated using a dataset (e.g. n=59 samples) (samples measured using both Nanostring and RT-PCR) that is divided into a training set (e.g. n=39 samples) and a cross-validation test set (e.g. n=20 samples) by random sampling, repeated 30 times (iteration I=30). For each iteration, the gene-wise conversion factors (intercept and slope) are obtained using linear regression models and are applied to adjust the RT-PCR data on the test set as follows:

Adjusted gene_{RT-PCR,zi}=β_{0,zi}+(β_{zi}×gene_{RT-PCR,zi})

Where adjusted gene_{RT-PCR,zi} is the adjusted RT-PCR mRNA level of gene (i), β_{0,zi} is the intercept of gene(i) in iteration z=1-30, and β_{zi} is the linear coefficient of gene(i) in iteration z=1-30.

The accuracy of conversion factors were evaluated by calculating the percentage error between the adjusted RT-PCR gene expression level for gene(i) and the NanoString gene expression level for gene(i):

Error (%)_{zi}=median{|(adjusted gene_{RT-PCR,zi}−gene_{NS,zi})/gene_{NS,zi}|*100}

Where adjusted gene_{RT-PCR,zi} is the adjusted RT-PCR mRNA expression level of gene(i) for the 20 test set samples, gene_{NS,zi} is the NanoString mRNA gene expression level of gene(i) in iteration z=1-30 for the 20 test set samples.

For each gene the conversion factors (intercept and slope) giving an error of <10% are averaged and equate to the final conversion factors:

$${β}_{0 , ci} = \frac{{\sum }_{j}^{n} {β}_{o , zt}}{n} and {β}_{ci} = \frac{{\sum }_{j}^{n} {β}_{zi}}{n}$$

Where β_{0,ci} and β_{ci} are the average of β_{0,zi} and β_{zi} conversion factors giving error of <10% for gene(i) in iteration z=1-30, and n is the number of coefficients averaged.

Finally, each of the gene-wise conversion factors β_{0,ci} and β_{ci} may be evaluated using an independent validation set (e.g. n=24) of samples measured by both NanoString and RT-PCR, as follows:

Adjusted gene expression levels=β_{0,ci}+(β_{ci}×gene_{RT-PCR,i})

Where β_{0,ci} and β_{ci} are the average of the conversion factors fiving accuracy of <10%. The performance may be assessed by comparing the resulting adjusted RT-PCR gene expression level with the NanoString gene expression level of the same gene.

### Verification of Conversion Factors and Adjusted Expression Levels for the Multi-Gene Prognostic Signature in a Validation Set.

An independent cohort of samples may be used for the validation of the gene-wise conversion factors. The averaged correction coefficients (gene-wise conversion factors) calculated for the training set may be imputed to adjust to Nanostring-derived gene expression levels. The size of the training set, test set and validation set, as well as the number of iterations and the chosen error threshold, are all illustrative values. The skilled person is readily able to apply a similar linear regression-based derivation of conversion factors using suitable values for size of the training set, test set and validation set, as well as the number of iterations and error threshold.

### Example 1—Derivation of the AIR-CIS Algorithm

The present inventors have developed an algorithm, AIR-CIS, which assesses four different gene expression modules for which there is laboratory and/or clinical evidence of an association with resistance to CDK4/6 inhibition: (i) E2F 22-gene signature (ii) CCNE1 (iii) RB loss and (iv) non-luminal intrinsic subtype. The inventors developed the AIR-CIS algorithm by bringing together the data on biomarkers observed to be related to response or resistance to CDK4/6 inhibition in several preclinical works and clinical studies reported to date as well as the inventors' own in-house data, and evaluated for its prevalence in samples from patients in POETIC to establish the final algorithm.

The development of the AIR-CIS algorithm was based on:
i. the relationships between components of the cell cycle that are dependent on CDK4/6 activity and its promotion of proliferation.
ii. evidence on relationships between these markers in laboratory model systems with de novo resistance or changes in their expression during acquisition of their resistance to CDK4/6 inhibition
iii. evidence on the relationship between the expression of these markers and response/resistance to CDK4/6 inhibition in clinical trials with preferential emphasis on data from pre-surgical studies of direct relevance to the POETIC-A design
iv. the prevalence/distribution of the biomarker in the POETIC data, specifically in the population to be studied in POETIC-A.

The evidence supports 4 molecular phenotypes as being able to differentiate sub-groups that have differing levels of sensitivity to CDK4/6 inhibitors in the overall population that is resistant to an AI. It is expressly contemplated that the signature may be applied to pre-treatment samples for adjuvant treatment of CDK4/6 as a first line of treatment. The populations identified by the individual phenotypes are overlapping but the inventors expressly envisage that the presence of any one of these individual phenotypes is sufficient to be designated as resistant.

The 4 phenotypic resistance modules are discussed below in terms of i) the known relationships between components of the cell cycle that are dependent on CDK4/6 activity and its promotion of proliferation, ii) evidence on relationships between these markers in laboratory model systems with de novo resistance or changes in their expression during acquisition of their resistance to CDK4/6 inhibition, and/or iii) evidence on the relationship between the expression of these markers and response/resistance to CDK4/6 inhibition in clinical trials with preferential emphasis on data from pre-surgical studies of direct relevance to the POETIC-A design.

### A. Non-Luminal Intrinsic Subtype (Intrinsic Subtype Classification)

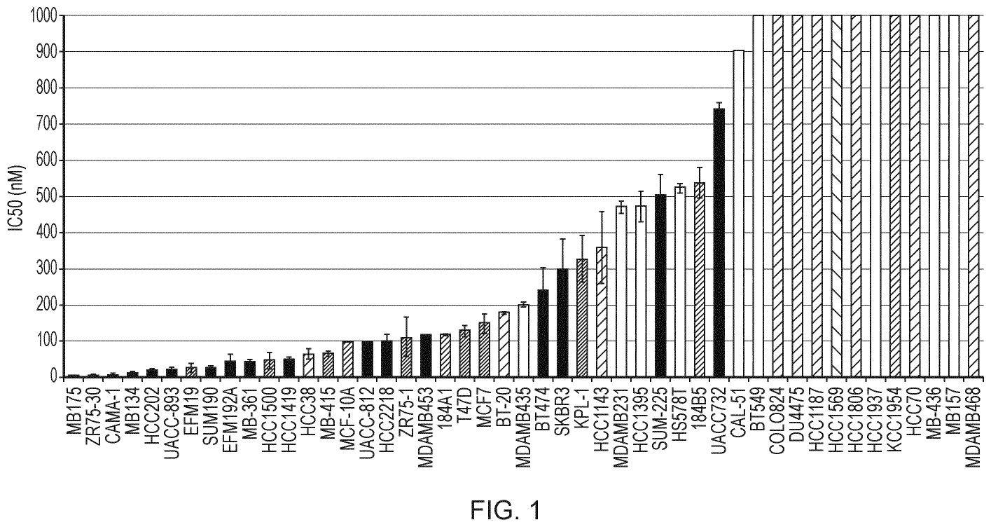

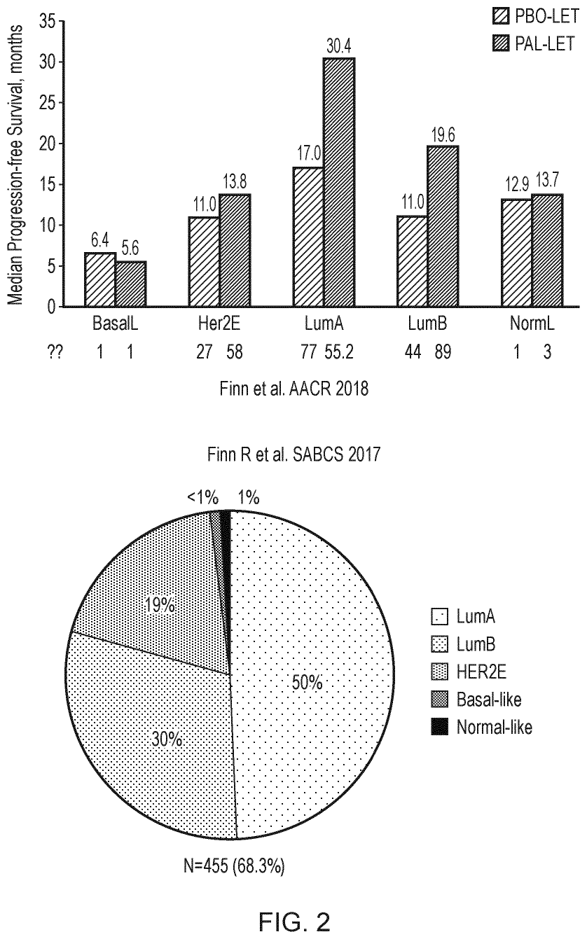

The effectiveness of CDK4/6 inhibition was assessed across a large panel of breast cancer cell lines by Finn et al (8) and was found to be almost exclusively restricted to the luminal intrinsic subtypes (FIG. 1). This has led to the development of the CDK4/6 inhibitors being focussed in the ER+ population. However, a significant proportion of ER+ breast cancers are non-luminal sub-types and these are enriched for patients with endocrine resistant disease as can be seen in POETIC. Clinical evidence that the non-luminal subtypes are also less responsive to CDK4/6 inhibition in combination with an AI is provided by the PALOMA-2 trial (NCT01740427) in which 20% of the population was found to be non-luminal (mostly HER2-enriched subtype) and showed minimal improvement in progression-free survival in contrast to the luminal subtypes (FIG. 2).

Intrinsic subtypes are commonly ascribed to that which has the strongest correlation to the respective nearest shrunken centroid. However, as described by Sorlie et al the confidence in this might be low in many tumours and above 95% confidence, when based on classical hierarchical clustering method correlation in only about 60% of the population she reported on. Additionally, the present inventors have shown that when the correlation with the respective centroid is similar between two intrinsic subtypes one frequently finds that a different subtype is ascribed when taking 2 core-cuts from the same tumour. In preferred embodiments, the AIR-CIS signature ascribes non-luminal subtype only when there is at least 95% confidence in the call based on the technically validated and statistically more robust PAM50 nearest-centroid method and at least 0.20 between the correlations with the two subtypes, i.e. sensitive (Luminal including Luminal A/B) vs. resistant (non-Luminal: Basal and HER2-Enriched).

### B. RB1 Deficiency (RB1 Loss)

RB1 has a pivotal role in cell cycle signalling downstream of CDK4/6. The present inventors' studies in model systems revealed RB-loss and RB-mutation to be responsible for acquired resistance to palbociclib. RB loss per se is uncommon in ER+ breast cancer but Malorni et al established an RB loss of function signature (RBsig) by identifying genes that correlated with E2F1 and E2F2 expression in breast cancers in TCGA. This was associated with worse relapse-free survival (RFS) in untreated and endocrine treated patients with ER+ breast cancer and differentiated palbociclib-resistant from palbociclib-sensitive cells. Bosco et al also created a signature from 53 genes that were deregulated with RB genetic loss and repressed upon RB activation and found this to be associated with worse DFS in 60 breast cancer patients. In the neoMONARCH presurgical study RB gene expression levels were significantly lower in non-responders than responders to single agent abemaciclib.

### C. High E2F Activity (E2F Signature)

Previous studies had demonstrated that higher expression associated with E2F activity is associated with endocrine resistance and hypothesized as being targetable with added CDK4/6 inhibition. Dr Arteaga's group has published 2 gene signatures associated with E2F activity that are associated with endocrine resistance in cell lines. The first of these is composed of 22 genes with an E2F motif but without a cell-cycle related GO annotation. It has been reported that the signature had a significant, modest correlation with Ki67 at baseline (r=0.29, p=0.014) but much stronger correlation with Ki67 measured in biopsies taken after 2 weeks' AI therapy (r=0.49, p=0.0026); the latter stronger correlation is the more relevant since it was made in tissue analogous to that to be assessed in the AIR-CIS in POETIC-A. This 22-gene signature is composed of genes that contained an E2F motif but did not have a cell-cycle related Gene Ontology (GO) annotation. The correlation was confirmed in a separate set of samples from patients treated with letrozole.

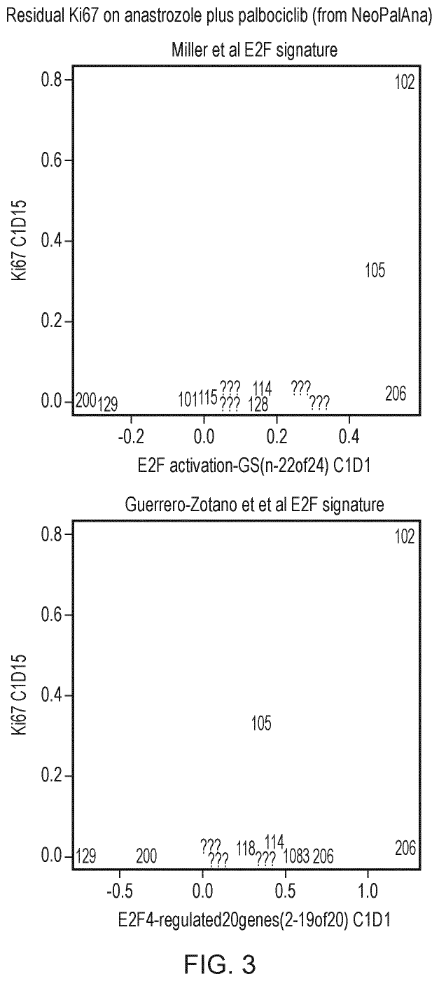

The NeoPalAna trial demonstrated that CDK4/6 inhibitor resistance was associated with non-luminal subtypes and persistent E2F-target gene expression. The present inventors were able to assess the two E2F signatures in anastrozole-treated tumours by accessing the supplementary data from the NeoPalAna study. Two of the three patients with highest expression of the Miller signature E2F activity were the only ones to show substantial continued Ki67 expression when treated with palbociclib plus anastrozole (FIG. 3). The Guerrero-Zotan signature identified only 1 of the 2 cases. The present inventors have therefore incorporated the Miller 22-gene signature as a module of in the AIR-CIS.

### D. High CCNE1 Activity (CCNE1 Expression)

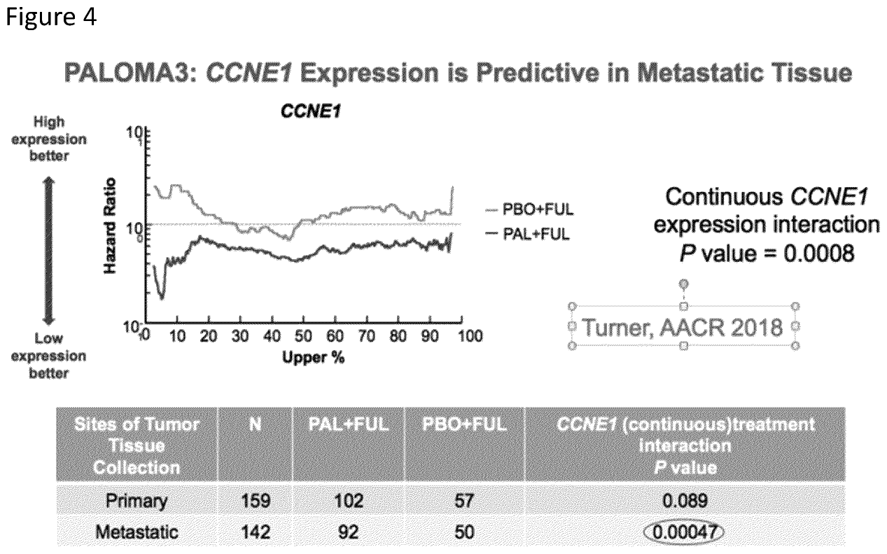

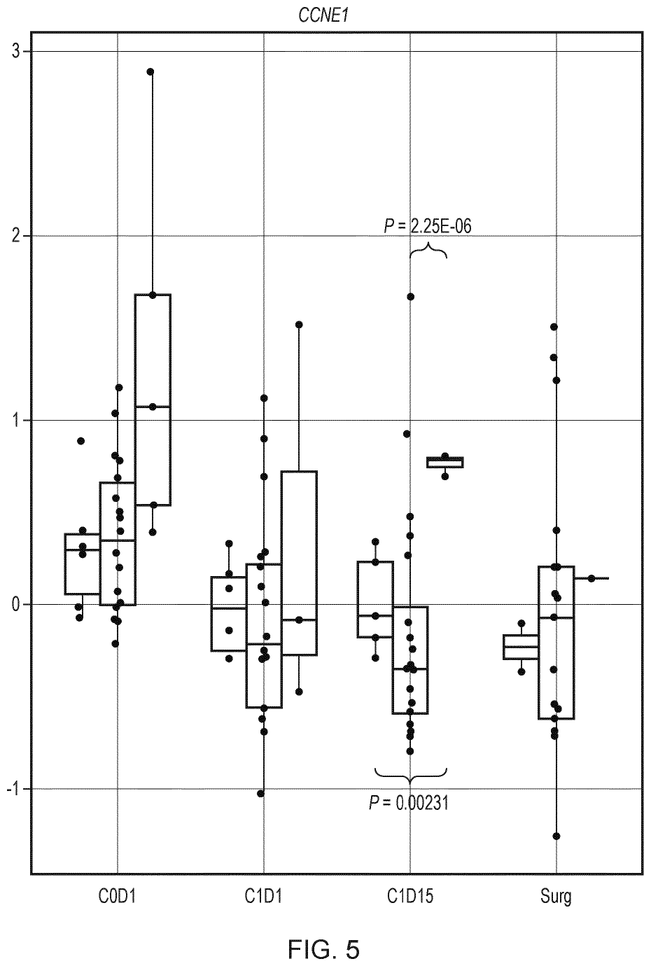

Several clinical and preclinical lines of evidence support high levels of CCNE1 being associated with CDK4/6 resistance. Cyclin-E1 (CCNE1) amplification was an alternative genetic change to RB loss leading to palbociclib resistance in our model systems as was overexpression independent of CN gain (25). Like RB1 loss, CCNE1 amplification is uncommon in ER+ breast cancer but in the PALOMA3 study (fulvestrant±palbociclib in ER+ advanced breast cancer) patients with high CCNE1 expression received significantly poorer benefit from added palbociclib; this relationship was considerably stronger when CCNE1 was measured in metastatic tissue. The greatest separation between those benefiting or not was for those in the highest 15-20% of CCNE1 expression (FIG. 4). In the neoadjuvant study of palbociclib added to anastrozole after initial treatment with the AI alone just 3 patients had resistance to the combined drugs. Uniquely in publications to date, analyses of gene expression were conducted on the samples after treatment with the AI alone (as is proposed in POETIC-A). As shown in FIG. 5, despite the very small number of samples CCNE1 expression was highly significantly higher in those resistant to the added palbociclib (p=2.25E−06).

Lastly in patients resistant to the AI in neoMonarch CCNE1 levels were non-significantly higher in the small number of patients resistant to added abemaciclib.

The inventors identified a panel of 81 genes (including 8 housekeeping genes) for further investigation of the AIR-CIS signature. The 81 genes are listed in Table 1

TABLE 1
81 genes of AIR-CIS signature (including 8 housekeeping genes):

| Name | Gene ID | Probe ID | Module(s) |
|---|---|---|---|
| ACTB | 60 | NM_001101.2:1685 | Housekeeping |
| ACTR3B | 57180 | NM_001040135.1:905 | Luminal |
| ANLN | 54443 | NM_018685.2:240 | Luminal |
| ARHGAP11A | 9824 | NM_199357.1:1820 | E2F |
| ATAD2 | 29028 | NM_014109.3:2648 | E2F |
| BAG1 | 573 | NM_004323.3:540 | Luminal |
| BCL2 | 596 | NM_000633.2:1525 | Luminal |
| BIRC5 | 332 | NM_001168.2:1215 | Luminal |
| BLVRA | 644 | NM_000712.3:485 | Luminal |
| C10ORF119 | 79892 | NM_024834.3:606 | E2F |
| CASP8AP2 | 9,994 | NM_012115.2:2040 | E2F |
| CCNB1 | 891 | NM_031966.2:710 | Luminal |
| CCNE1 | 898 | NM_001238.1:1155 | CCNE1 and |
| | | | Luminal |
| CDC20 | 991 | NM_001255.1:915 | Luminal |
| CDC6 | 990 | NM_001254.3:1655 | Luminal |
| CDH3 | 1001 | NM_001793.3:2005 | Luminal |
| CENPF | 1063 | NM_016343.3:9260 | Luminal |
| CEP55 | 55165 | NM_018131.3:570 | Luminal |
| CLSPN | 63967 | NM_022111.2:442 | E2F |
| CXXC5 | 51523 | NM_016463.5:1630 | Luminal |
| DCK | 1633 | NM_000788.2:310 | E2F |
| DNAJC9 | 23234 | NM_015190.3:530 | E2F |
| EGFR | 1956 | NM_005228.3:2760 | Luminal |
| ERBB2 | 2064 | NM_004448.2:2405 | Luminal |
| ESR1 | 2099 | NM_000125.2:1595 | Luminal |
| EXO1 | 9156 | NM_006027.3:820 | Luminal |
| FANCD2 | 2177 | NM_033084.3:260 | E2F |
| FBXO5 | 26271 | NM_012177.3:898 | E2F |
| FGFR4 | 2264 | NM_002011.3:1002 | Luminal |
| FKBP5 | 2289 | NM_001145775.1:540 | E2F |
| FOXA1 | 3169 | NM_004496.2:280 | Luminal |
| FOXC1 | 2296 | NM_001453.1:1530 | Luminal |
| GPR160 | 26996 | NM_014373.1:760 | Luminal |
| GRB7 | 2886 | NM_005310.2:1010 | Luminal |
| GUSB | 2990 | NM_000181.1:1350 | Housekeeping |
| H2AFZ | 3015 | NM_002106.3:111 | E2F |
| KIAA0101 | 9768 | NM_014736.5:1025 | E2F |
| KIF2C | 11004 | NM_006845.2:1020 | Luminal |
| KPNB1 | 3837 | NM_002265.4:2580 | E2F |
| KRT14 | 3861 | NM_000526.3:1365 | Luminal |
| KRT17 | 3872 | NM_000422.1:1230 | Luminal |
| KRT5 | 3852 | NM_000424.2:130 | Luminal |
| MAPT | 4137 | NM_016835.3:1425 | Luminal |
| MDM2 | 4193 | NM_006878.2:280 | Luminal |
| MELK | 9833 | NM_014791.2:365 | Luminal |
| MIA | 8190 | NM_006533.1:265 | Luminal |
| MKI67 | 4288 | NM_002417.2:2005 | Luminal |
| MLPH | 79083 | NM_024101.4:1695 | Luminal |
| MMP11 | 4320 | NM_005940.3:702 | Luminal |
| MRPL19 | 9801 | NM_014763.3:385 | Housekeeping |
| MYBL2 | 4605 | NM_002466.2: 675 | Luminal |
| MYC | 4609 | NM_002467.3:1615 | Luminal |
| NAT1 | 9 | NM_000662.4:0 | Luminal |
| NDC80 | 10403 | NM_006101.1:90 | Luminal |
| (alias KNTC2) | | | |
| NUF2 | 83540 | NM_145697.1:215 | Luminal |
| (alias CDCA1) | | | |
| NUP62 | 23636 | NM_016553.3:457 | E2F |
| ORC6L | 23594 | NM_014321.2:580 | Luminal |
| PGR | 5241 | NM_000926.2:3165 | Luminal |
| PHGDH | 26227 | NM_006623.2:505 | Luminal |
| PSMC4 | 5704 | NM_006503.2:300 | Housekeeping |
| PTTG1 | 9232 | NM_004219.2:202 | Luminal |
| PUM1 | 9698 | NM_001020658.1:640 | Housekeeping |
| RANBP1 | 5092 | NM_002882.2:380 | E2F |
| RB1 | 5925 | NM_000321.1:2110 | RB1 |
| RET | 5979 | NM_020630.4:2911 | E2F |
| RPLP0 | 6175 | NM_001002.3:250 | Housekeeping |
| RRM2 | 6241 | NM_001034.1:490 | Luminal |
| SF3A1 | 10291 | NM_005877.4:1485 | Housekeeping |
| SFRP1 | 6422 | NM_003012.3:1320 | Luminal |
| SFRS1 | 6426 | NM_006924.4:2370 | E2F |
| SFRS10 | 6434 | NM_001191009.2:450 | E2F |
| SFRS7 | 6432 | NM_001031684.2:339 | E2F |
| SLC39A6 | 25800 | NM_012319.2:1580 | Luminal |
| SNRPD1 | 6632 | NM_006938.2:1204 | E2F |
| STMN1 | 3925 | NM_203401.1:478 | E2F |
| TFRC | 7037 | NM_003234.1:1220 | Housekeeping |
| TMEM45B | 120224 | NM_138788.3:730 | Luminal |
| TMPO | 7112 | NM_001032284.1:576 | E2F |
| TYMS | 7298 | NM_001071.1:395 | Luminal |
| UBE2C | 11065 | NM_007019.2:445 | Luminal |
| UBE2T | 29089 | NM_014176.1:50 | Luminal |

Gene ID refers to the NCBI Gene ID available at https://www.ncbi.nlm.nih.gov/gene on 26 Aug. 2020. The Gene ID record for each of the human genes in table 1 is expressly incorporated herein by reference in its entirety.

### Example 2—Distribution of the Resistance Modules in the Target Population

The four phenotypic resistance modules are discussed below in terms of iv) the prevalence/distribution of the biomarker in the POETIC data, specifically in the population to be studied in POETIC-A.

Using the data from an RNA panel of 81 genes (including 8 housekeeper genes—see Table 1) that had been assessed on the Nanostring platform in relevant tumours from the POETIC trial after 2 weeks' AI the inventors have been able to establish cut-offs for the genes/signatures that underpin each phenotypic module that can be applied to unknown samples by reference to a set of housekeeping genes. This Nanostring data was used to calculate the gene expression of each of the four modules from 52 on-AI samples from the POETIC trial with Ki67B≥20% and on-treatment Ki672w≥8%, i.e. the subpopulation of interest for POETIC-A.

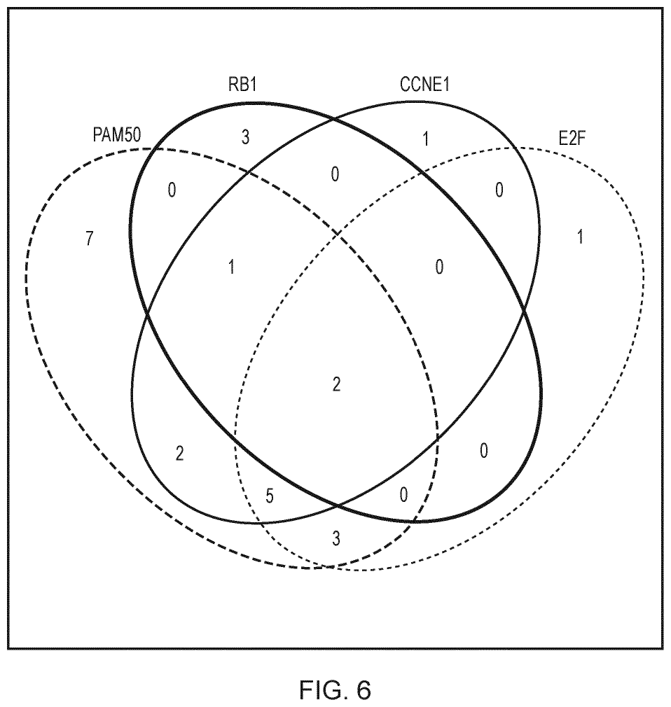

Of the 52 patients, 27 were deemed to be CDK4/6 inhibitor-sensitive and 25 to be resistant according to the signature. Resistance according to the modular components and the overlap between the modules in this respect is shown in FIG. 6. Overall, using the stated cut-offs for each module at least one of them is present and indicative of resistance in approximately 50% of the population. There is major overlap between the modules across the population. Of particular note 20/25 of the patients deemed to be resistant by one of CCNE1, RB1 or E2F were also identified as such by the PAM50-non-luminal module alone.

The present inventors propose that the AIR-CIS signature using all 4 modules continues to be the primary predictive measure of resistance but that as secondary analyses POETIC-A will be able to evaluate the predictive value of (i) non-luminal status alone and (ii) non-luminal status accompanied by positivity in at least one of the other 3 modules. In the series assessed that latter group composed 13/52 (25%) of the group that represents the proposed POETIC-A randomised population.

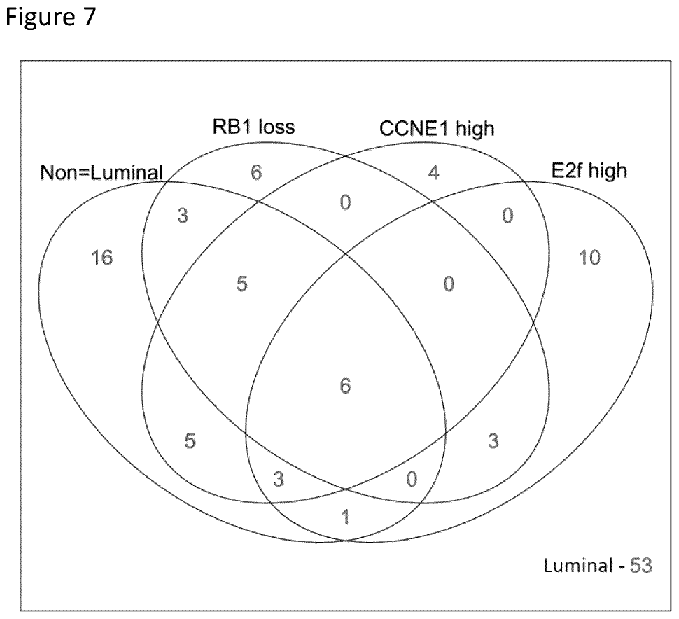

The present inventors have additionally determined the expression of the individual genes/modules and their overlap and the distribution of the AIR-CIS categories in a further 63 samples also from the POETIC Trial and having tumours with Ki67B≥20% and on-treatment Ki672w≥8%. These further 63 samples were used to confirm and extend the data described above. Of the total of 115 tumours 53 tumours were categorised as sensitive and 62 as resistant (i.e. at least one of non-Luminal, low RB, high E2F, high CCNE1). The inventors were able to determine the prevalence and overlap of each of these gene modules in the cases in the POETIC trial with pre-treatment Ki67≥20% and 2-week Ki67≥8%. Shown in FIG. 7 is a Venn diagram of 115 tumours with 53 tumours considered as sensitive and 62 as resistant (i.e. at least one of non-Luminal, low RB, high E2F, high CCNE1).

Control samples, created from pooling 5 samples previously categorised as AIR-CIS sensitive or resistant, were also included and the variability of the constituent components of these was assessed in a series of technical replicates.

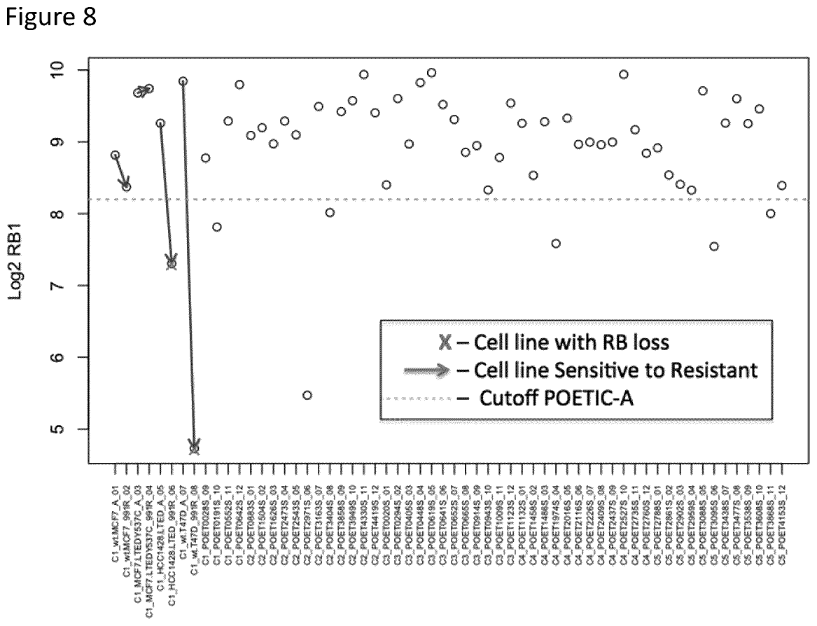

Based on previous work with cell lines and samples from the POETIC trial, the inventors have newly identified the cut-off value for RB1 gene expression that corresponds to loss or very low expression of RB1. For AIR-CIS the present inventors have identified the level of gene expression in our cell lines with either RB loss or mutation and created a cut-off for RB expression in which c.15% of the cases are low expressers using POETIC samples (FIG. 8).

The present inventors have recently found that in 133 ER+/HER2− tumours from the POETIC trial on-treatment expression of this E2F signature correlates with residual Ki67 at 2 weeks similar to the earlier study: r=0.45, p=5.4E−07 (unpublished).

Using samples from the POETIC trial, the present inventors have newly identified the cut-off value for CCNE1 gene expression that corresponds to high or very low expression of CCNE1.

As a result of this analysis, the present inventors will apply the following cut-offs to define following modules for POETIC-A tumours: E2F>/=9.392 or E2F>/=9.4462 (average log 2 expression of E2F signature genes); CCN1E>/=8.264 or CCN1E>/=7.9596 (log 2); RB deficiency</=8.4068 or</=8.4332 (log 2). The laboratory standard operating procedure will contain information on these cut-offs and also QC acceptability/rejection criteria for individual batches/samples.

### Prophetic Example 3—The POETIC-A Trial

The POETIC-A trial seeks to confirm whether the four molecular phenotypes are able to differentiate sub-groups that have differing levels of sensitivity to CDK4/6i in the overall population that is resistant to an AI.

A sample will be classified as resistant if at least one of the four component modules call resistant phenotype which are as follows: non-luminal subtype or low RB1 or high CCNE1 or high E2F score. A sample will be classed as sensitive if all four component module calls are sensitive as follows: luminal subtype and high RB1 and low CCNE1 and low E2F score.

In POETIC-A, a tumour will be considered as AIR-CIS resistant if classified as non-luminal according to the PAM50 Bioclassifier. A tumour will be classified as AIR-CIS resistant if the RB gene expression as measured by Nanostring is ≤8.4068 or ≤8.4332 (log 2). The inventors will apply the E2F signature composed of genes that did not have a cell-cycle related GO annotation. A tumour will be classified as AIR-CIS resistant if the E2F activity signature score, as measured by Nanostring, is ≥9.392 or ≥9.4462 (log 2) (average log 2 expression of E2F signature genes). Using on-AI samples from the POETIC trial with Ki67B≥20% and on-treatment Ki672w≥8%, the present inventors have identified a cut-off at ≥8.264 or at ≥7.9596 (log 2) for CCNE1 as measured by Nanostring, to classify a tumour as AIR-CIS resistant.

### Prophetic Example 4—The PALLET Trial

The PALLET trial is a phase II, randomised study evaluating the biological and clinical effects of the combination of palbociclib (a CDK4/6 inhibitor) with letrozole (an aromatase inhibitor) as neoadjuvant therapy in post-menopausal women with ER+ primary breast cancer.

### Example 5—Generation of Reduced Size Classifiers to Capture Luminal vs Non-Luminal Cases

The present inventors set out to generate a reduced gene list to capture Luminal vs non-Luminal cases. Cases that were prototypical within the subgroup of AI-resistant tumours were included in this analysis (i.e. cases that show close similarity to both resistant (non-Luminal) and sensitive (Luminal) subtypes were excluded from this analysis) in order to obtain a more precise error rate.

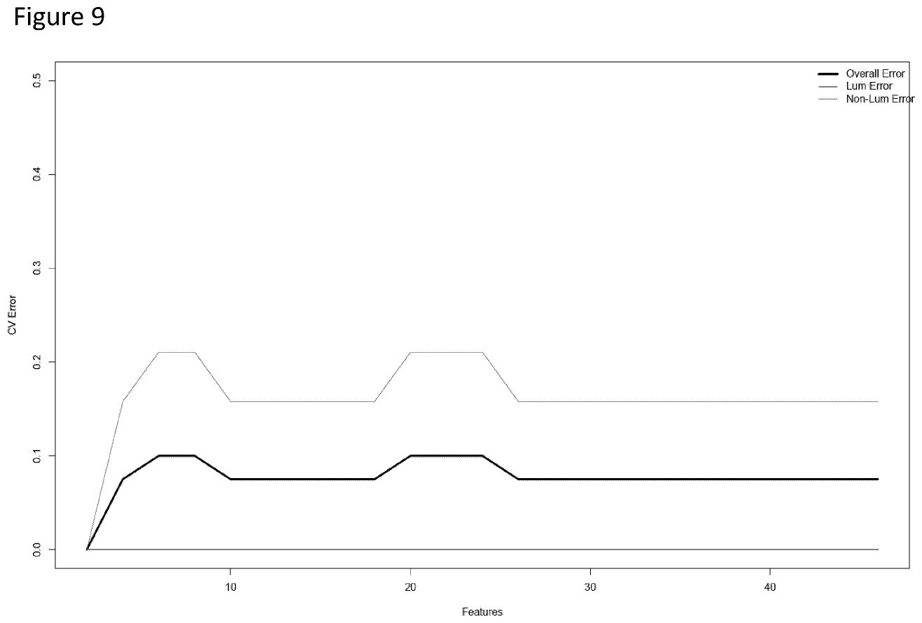

The overall, sensitive (Luminal) and resistant (non-Luminal) error rates for each total number of genes are shown in Table 2 and FIG. 9.

TABLE 2
Error rates for Luminal vs non-Luminal classification
according to number of genes analysed.

| nGenes | nGenes | Overall | Sensitive | Resistant (Non Luminal) |
|---|---|---|---|---|
| per class | (Total) | Error | error rate | error rate |
| 1 | 2 | 0 | 0 | 0 |
| 2 | 4 | 0.075 | 0 | 0.157894737 |
| 3 | 6 | 0.1 | 0 | 0.210526316 |
| 4 | 8 | 0.1 | 0 | 0.210526316 |
| 5 | 10 | 0.075 | 0 | 0.157894737 |
| 6 | 12 | 0.075 | 0 | 0.157894737 |
| 7 | 14 | 0.075 | 0 | 0.157894737 |
| 8 | 16 | 0.075 | 0 | 0.157894737 |
| 9 | 18 | 0.075 | 0 | 0.157894737 |
| 10 | 20 | 0.1 | 0 | 0.210526316 |
| 11 | 22 | 0.1 | 0 | 0.210526316 |
| 12 | 24 | 0.1 | 0 | 0.210526316 |
| 13 | 26 | 0.075 | 0 | 0.157894737 |
| 14 | 28 | 0.075 | 0 | 0.157894737 |
| 15 | 30 | 0.075 | 0 | 0.157894737 |
| 16 | 32 | 0.075 | 0 | 0.157894737 |
| 17 | 34 | 0.075 | 0 | 0.157894737 |
| 18 | 36 | 0.075 | 0 | 0.157894737 |
| 19 | 38 | 0.075 | 0 | 0.157894737 |
| 20 | 40 | 0.075 | 0 | 0.157894737 |
| 21 | 42 | 0.075 | 0 | 0.157894737 |
| 22 | 44 | 0.075 | 0 | 0.157894737 |
| 23 | 46 | 0.075 | 0 | 0.157894737 |

As demonstrated in Table 2 and FIG. 9, use of a total number of genes ranging between 2 and 46 (between 1 and 23 per class) results in an overall error rate of ≤1. Use a total of 4 genes (2 per class) can classify a case as Luminal vs non-Luminal with an overall error rate of 0.075.

Table 3 lists the genes that were included in each analysis and the centroids for luminal and non-luminal, respectively, where n=the total number of genes analysed. It is specifically contemplated herein that the lists of genes provided in Table 3 may be used as the luminal module within the AIR-CIS algorithm to classify a case as Luminal vs non-Luminal. It is specifically contemplated herein that 4-gene list provided in Table 3 (i.e. ANLN, ESR1, PGR and SLC39A6) may be used as the luminal module within the AIR-CIS algorithm to classify a case as Luminal vs non-Luminal.

TABLE 3
Reduced size gene lists for Luminal vs non-Luminal classification

| | Centroids | Centroids |
|---|---|---|
| | 1 (Luminal- | 2 (Non-Luminal- |
| Combination | Sensitive) | Resistant) |
| Genes (n = 2) | | |
| ESR1 | 0.724924673 | −0.293827849 |
| SLC39A6 | 2.236554031 | 1.642473045 |
| Genes (n = 4) | | |
| ANLN | −0.436597966 | −0.171060579 |
| ESR1 | 0.724924673 | −0.293827849 |
| PGR | −0.14811616 | −0.58065462 |
| SLC39A6 | 2.236554031 | 1.642473045 |
| Genes (n = 6) | | |
| ANLN | −0.436597966 | −0.171060579 |
| BCL2 | 0.214783191 | −0.257430908 |
| ESR1 | 0.724924673 | −0.293827849 |
| PGR | 0.243228161 | −0.14811616 |
| PHGDH | −0.008631878 | 0.414898718 |
| SLC39A6 | 2.236554031 | 1.642473045 |
| Genes (n = 8) | | |
| ANLN | −0.436597966 | −0.171060579 |
| BCL2 | 0.214783191 | −0.257430908 |
| CENPF | 0.261621563 | 0.531497672 |
| ESR1 | 0.724924673 | −0.293827849 |
| PGR | 0.243228161 | −0.14811616 |
| PHGDH | −0.391826227 | −0.008631878 |
| RRM2 | 0.59104305 | 0.842673949 |
| SLC39A6 | 2.236554031 | 1.642473045 |
| Genes (n = 10) | | |
| ANLN | −0.436597966 | −0.171060579 |
| BCL2 | 0.214783191 | −0.257430908 |
| CDC20 | −0.347286987 | −0.09561991 |
| CDH3 | −0.522006879 | −0.173025486 |
| CENPF | 0.261621563 | 0.531497672 |
| ESR1 | 0.724924673 | −0.293827849 |
| PGR | 0.243228161 | −0.14811616 |
| PHGDH | −0.391826227 | −0.008631878 |
| RRM2 | 0.363376998 | 0.59104305 |
| SLC39A6 | 2.236554031 | 1.642473045 |
| Genes (n = 12) | | |
| ANLN | −0.436597966 | −0.171060579 |
| BCL2 | 0.214783191 | −0.257430908 |
| CDC20 | −0.347286987 | −0.09561991 |
| CDH3 | −0.522006879 | −0.173025486 |
| CENPF | 0.017447942 | 0.261621563 |
| ESR1 | 0.724924673 | −0.293827849 |
| PGR | 0.243228161 | −0.14811616 |
| PHGDH | −0.391826227 | −0.008631878 |
| PTTG1 | 0.028806637 | 0.23337021 |
| RRM2 | 0.363376998 | 0.59104305 |
| SLC39A6 | 2.236554031 | 1.642473045 |
| UBE2C | 0.060331049 | 0.318978765 |
| Genes (n = 14) | | |
| ANLN | −0.436597966 | −0.171060579 |
| BCL2 | 0.214783191 | −0.257430908 |
| CDC20 | −0.574985771 | −0.347286987 |
| CDH3 | −0.522006879 | −0.173025486 |
| CENPF | 0.017447942 | 0.261621563 |
| ESR1 | 0.724924673 | −0.293827849 |
| FOXA1 | 0.774898643 | 0.173550394 |
| MLPH | 0.825757051 | 0.357073665 |
| PGR | 0.243228161 | −0.14811616 |
| PHGDH | −0.391826227 | −0.008631878 |
| PTTG1 | 0.028806637 | 0.23337021 |
| RRM2 | 0.363376998 | 0.59104305 |
| SLC39A6 | 2.236554031 | 1.642473045 |
| UBE2C | 0.060331049 | 0.318978765 |

It is evident from Table 3 that the gene lists are concentric. For instance, the genes ESR1 and SLC39A6 which constitute the n=2 gene list are present in the n=4, 6, 8, 10, 12 and 14 gene lists. Similarly, the genes ANLN, ESR1, PGR and SLC39A6 which constitute the n=4 gene list are present in the n=6, 8, 10, 12 and 14 gene lists. This confirms the significance of the expression of such genes in classifying a sample as sensitive (Luminal) or resistant (non-Luminal) to AI.

### Example 6—Generation of Reduced Size Classifiers to Capture the E2F Signature

The present inventors have developed a gene list of 22 genes to classify the E2F signature. These 22 genes are listed in Table 4.

TABLE 4
Gene list of 22 genes for E2F signature.

| | |
|---|---|
| ARHGAP11A | KIAA0101 |
| ATAD2 | KPNB1 |
| C10ORF119 | NUP62 |
| CASP8AP2 | RANBP1 |
| CLSPN | RET |
| DCK | SFRS1 |
| DNAJC9 | SFRS10 |
| FANCD2 | SFRS7 |
| FBXO5 | SNRPD1 |
| FKBP5 | STMN1 |
| H2AFZ | TMPO |

The present inventors developed gene combinations with less than 10%, 5% or 1% misclassification rate (5%, 2.5% and 0.5% Type I and II errors respectively). These gene combinations are shown in Tables 5, 6, and 7 respectively. It is expressly contemplated herein that any of these gene combinations may be used as the E2F module in the AIR-CIS algorithm to classify the E2F signature. It is expressly contemplated herein that the 5-gene signature (SFRS1, DNAJC9, FBXO5, DCK, and TMPO) may be used as the E2F module in the AIR-CIS algorithm to classify the E2F signature.

TABLE 5
Gene combinations for E2F signature with less than 10%
misclassification rates (5% Type I and II errors)
Genes (n = 3)

| | | |
|---|---|---|
| SFRS1 | DNAJC9 | FBXO5 |

TABLE 6
Gene combinations for E2F signature with less than 5% misclassification rates (2.5% Type I and II errors)
Genes (n = 4)

| | | | |
|---|---|---|---|
| SFRS1 | DNAJC9 | FBXO5 | SNRPD1 |

Genes (n = 5)

| | | | | |
|---|---|---|---|---|
| SFRS1 | DNAJC9 | FBXO5 | DCK | TMPO |

Genes (n = 6)

| | | | | | |
|---|---|---|---|---|---|
| CLSPN | RET | ARHGAP11A | STMN1 | ATAD2 | KPNB1 |
| CLSPN | RET | ARHGAP11A | STMN1 | SNRPD1 | RANBP1 |
| CLSPN | RET | ARHGAP11A | STMN1 | ATAD2 | RANBP1 |
| CLSPN | RET | ARHGAP11A | STMN1 | SNRPD1 | TMPO |
| CLSPN | RET | ARHGAP11A | STMN1 | SNRPD1 | FBXO5 |
| CLSPN | RET | ARHGAP11A | STMN1 | ATAD2 | SNRPD1 |
| CLSPN | RET | ARHGAP11A | STMN1 | ATAD2 | KIAA0101 |
| CLSPN | RET | ARHGAP11A | STMN1 | ATAD2 | FBXO5 |
| CLSPN | RET | ARHGAP11A | SNRPD1 | FBXO5 | TMPO |
| CLSPN | RET | ARHGAP11A | ATAD2 | SNRPD1 | FBXO5 |
| CLSPN | RET | STMN1 | SNRPD1 | TMPO | KIAA0101 |
| CLSPN | RET | STMN1 | ATAD2 | SNRPD1 | FBXO5 |
| ARHGAP11A | ATAD2 | CASP8AP2 | NUP62 | SFRS1 | SFRS10 |

TABLE 7
Gene combinations for E2F signature with less than 1%
misclassification rates (0.5% Type I and II errors)
Genes (n = 7)

| | | | | | | |
|---|---|---|---|---|---|---|
| FKBP5 | SFRS1 | SFRS10 | KIAA0101 | CASP8AP2 | DNAJC9 | FBXO5 |

Genes (n = 8)

| | | | | | | | |
|---|---|---|---|---|---|---|---|
| RET | CLSPN | ARHGAP11A | SNRPD1 | STMN1 | FBXO5 | TMPO | RANBP1 |
| RET | CLSPN | ARHGAP11A | SNRPD1 | STMN1 | FBXO5 | ATAD2 | RANBP1 |
| RET | CLSPN | ARHGAP11A | STMN1 | FBXO5 | TMPO | ATAD2 | SFRS1 |
| RET | CLSPN | ARHGAP11A | STMN1 | FBXO5 | ATAD2 | SFRS1 | C10ORF119 |
| RET | CLSPN | ARHGAP11A | SNRPD1 | STMN1 | FBXO5 | TMPO | ATAD2 |
| RET | CLSPN | ARHGAP11A | SNRPD1 | STMN1 | FBXO5 | ATAD2 | C10ORF119 |
| CLSPN | FBXO5 | FKBP5 | KIAA0101 | SFRS1 | DCK | SFRS10 | DNAJC9 |
| FBXO5 | FKBP5 | KIAA0101 | SFRS1 | DCK | SFRS10 | CASP8AP2 | DNAJC9 |
| FBXO5 | FKBP5 | KIAA0101 | SFRS1 | DCK | SFRS10 | C10ORF119 | DNAJC9 |
| FBXO5 | FKBP5 | KIAA0101 | SFRS1 | SFRS10 | CASP8AP2 | DNAJC9 | SFRS7 |
| FBXO5 | FKBP5 | KIAA0101 | SFRS1 | SFRS10 | CASP8AP2 | DNAJC9 | NUP62 |

The present inventors found that the 5-gene list (SFRS1, DNAJC9, FBXO5, DCK, and TMPO) had only 1 resistant misclassification and a high Pearson correlation to the original result (>0.9). The 5-gene list advantageously provides a compact yet accurately predictive gene signature that may be employed as the E2F module for the AIR-CIS algorithm.

Following the work described above to identify minimal, compact signatures that reduce the number of genes that required gene expression measurement while still exhibiting minimal misclassification error, the present inventors have identified a more compact gene signature for use in the AIR-CIS algorithm. In this compact AIR-CIS signature the two modules that comprised the greatest number of genes in the 81-gene set (Table 1), namely the luminal vs. non-luminal module (50 genes) and the E2F module (22 genes), have been significantly reduced in size to n=4 genes (compact luminal vs. non-luminal; see Table 3) and n=5 genes (compact E2F; see Table 6). Accordingly, the compact AIR-CIS signature may comprise or consist of the following genes, grouped by modules:
(i) Genes of the Luminal vs. non-luminal compact module: ANLN, ESR1, PGR and SLC39A6;
(ii) Genes of the E2F compact module: SFRS1, DNAJC9, FBXO5, DCK, and TMPO;
(iii) RB1 module: RB1; and
(iv) CCNE1 module: CCNE1.

Therefore, in certain embodiments, the AIR-CIS signature may comprise or consist of the following “compact” 11-gene set: ANLN, ESR1, PGR, SLC39A6, SFRS1, DNAJC9, FBXO5, DCK, TMPO, RB1 and CCNE1, optionally further comprising or further consisting of one or more (e.g. 2, 3, 4, 5, 6, 7 or 8) housekeeping genes. The compact AIR-CIS signature advantageously reduces the number of genes of which gene expression must be measured, thereby saving time and resources. In certain embodiments, the total number of genes used in the AIR-CIS signature and in the methods and systems of the present invention may be not more than 50, such as not more than 40, not more than 30, not more than 25, not more than 24, not more than 23, not more than 22, not more than 21, not more than 20, not more than 19, not more than 18, not more than 17, not more than 16, not more than 15, not more than 14, not more than 13, not more than 12 or even not more than 11.

### Example 7—Additional Validation of AIR-CIS Performance

The present inventors performed additional gene expression profiling experiments for assessing the prevalence of AIR-CIS defined resistance modules in the target population, specifically post-menopausal women with ER+ HER2− tumours, with baseline Ki67>20% and Ki67 after 2 weeks aromatase inhibitor (AI) treatment >8%. These patients would be considered as showing AI resistance, at higher risk of recurrence and requiring additional treatment, such as CDK4/6 inhibitors. These additional 96 post-2 wk AI tumour samples included 53 patients in the treatment arm from the POETIC trial, 27 patients from the PALLET trial and 16 sensitive/resistant controls taken from the POETIC trial.

The sensitive/resistant controls were added to each run (max 12 samples per run on the Nanostring platform in this case) to ensure validity of the sensitive/resistant calls for the four modules in the test patients. Sensitive and resistant controls were correctly classified in the 8 runs performed, confirming the robustness of the assay.

Among the additional 80 tested patients (53 from POETIC and 27 from PALLET), 40 were classified as sensitive and 40 Resistant to CDK4/6 inhibitor using the AIR-CIS algorithm respectively.

Below is the breakdown of the AIR-CIS profiles of these 80 AI-resistant tumours:
13 with RB1 loss
20 with high CCNE1 expression
25 with high E2F targets expression

25 with non-luminal subtype

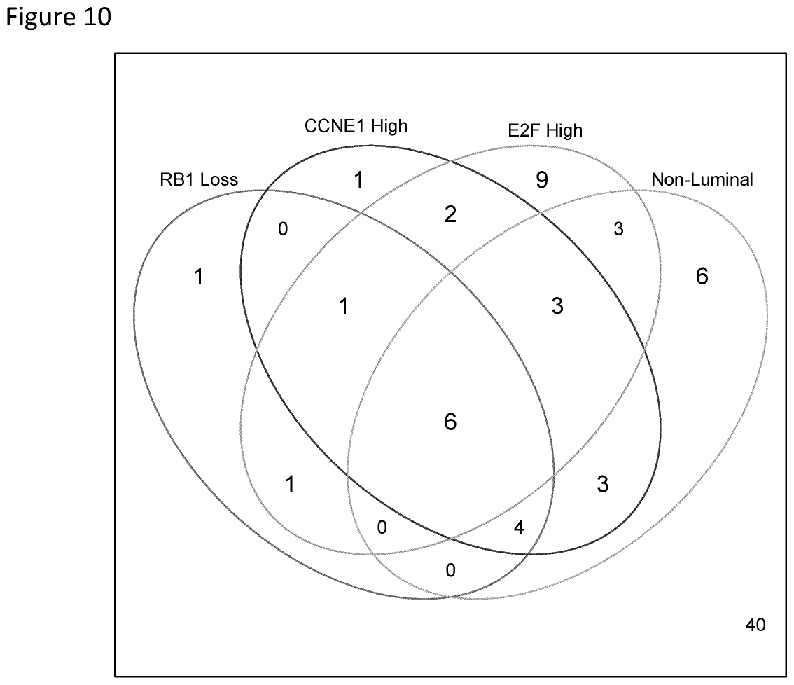

The following is a break-down in terms of the AI-resistant tumours with overlapping of CDK4/6 resistance modules as defined by AIR-CIS (see also FIG. 10):
6 with four resistant modules
8 with three resistant modules
9 with 2 resistant modules
17 with 1 resistant module

Within the 27 samples from PALLET study (Johnston et al., 2019—see ref. [9]), 11 were from the arm B of the study in which these tumours were treated with palbociclib plus letrozole to another 14 weeks; 7 of these we have proliferation biomarker Ki67 data at 14 wk. Based on this clinically accepted Ki67-based definition (14 wk Ki67>2.7%), only one patient would be considered as Palbociclib (PALBO) resistant with 14 wk Ki67 at 6.3%. This reflects the fact that the PALLET trial was not optimally designed for the assessment of AIR-CIS predictive performance. There are too few resistant patients within this group to provide statistical power for such assessment and enrichment of the sensitive luminal subtypes compared to what would be expected from AI resistant tumours (25 of 27 PALLET tumours classified as luminal compared to 30 out of 53 POETIC tumours). Nevertheless, 4/7 AIR-CIS calls were concordant with the post-treatment tumour proliferation rate marker (Ki67) defined response categories. This shows that, notwithstanding the limitations inherent in the PALLET sample numbers and break-down of resistant/sensitive, the AIR-CIS performance robustly tracks that seen with the POETIC trial data.

### Assessing the Technical Robustness of AIR-CIS Defined Resistance Modules on Individual Biopsy Sample

The AIR-CIS panel includes gene expression data to determine intrinsic subtype and the luminal (sensitive) and non-luminal (resistant) subtype. This is one of the most important modules in AIR-CIS. As part of the validation of the assay, “gold standard” intrinsic subtyping (as defined by the commercial Prosigna assay) was performed on the 80 tested POETIC/PALLET patients and the present inventors compared this gold standard result to the intrinsic subtype definition of the AIR-CIS panel.

The present inventors hypothesised that, if suboptimal, the parameters and algorithm could be refined to improve the precision of AIR-CIS algorithm to define Luminal/non-luminal on an individual sample (rather than batch of 10 samples in single run) and also to calculate a calibration factor by including known references in order to standardise these calls across different batches of samples.

The present inventors performed the Nanostring BC360 panel on the additional tested samples, and 78 patients received high confidence intrinsic subtyping with the “gold standard” BC360 assay (which is equivalent to the commercial Prosigna subtype). With the AIR-CIS algorithm of the present invention, there was 80% concordance with intrinsic subtype and 89% concordance with luminal/non-luminal classification. Using this new data, the present inventors were able to improve the precision of the assay to 95% for both intrinsic subtype and luminal/non-luminal classification. In addition, the present inventors were able to use this “gold standard” intrinsic subtype data on other sources of gene expression data including other Nanostring datasets and RNA-seq data in order to achieve >90% concordance of intrinsic subtyping calls in other data types. This will permit simulation of the AIR-CIS assay on >300 additional POETIC patients with RNA-seq gene expression data.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

1. Goetz M P, Toi M, Campone M, et al: MONARCH 3: Abemaciclib As Initial Therapy for Advanced Breast Cancer. J Clin Oncol 35:3638-3646, 2017
2. Rugo H S, Finn R S, Dieras V, et al: Palbociclib plus letrozole as first-line therapy in estrogen receptor-positive/human epidermal growth factor receptor 2-negative advanced breast cancer with extended follow-up. Breast Cancer Res Treat 174:719-729, 2019
3. Parker J S, Mullins M, Cheang M C, et al: Supervised risk predictor of breast cancer based on intrinsic subtypes. J Clin Oncol 27:1160-7, 2009
4. Herrera-Abreu M T, Palafox M, Asghar U, et al: Early Adaptation and Acquired Resistance to CDK4/6 Inhibition in Estrogen Receptor-Positive Breast Cancer. Cancer Res 76:2301-13, 2016
5. Turner N C, Liu Y, Zhu Z, et al: Cyclin E1 Expression and Palbociclib Efficacy in Previously Treated Hormone Receptor-Positive Metastatic Breast Cancer. J Clin Oncol 37:1169-1178, 2019
6. Miller T W, Balko J M, Fox E M, et al: ERalpha-dependent E2F transcription can mediate resistance to estrogen deprivation in human breast cancer. Cancer Discov 1:338-51, 2011
7. Ma, C. X., et al. (2017). “NeoPalAna: Neoadjuvant Palbociclib, a Cyclin-Dependent Kinase 4/6 Inhibitor, and Anastrozole for Clinical Stage 2 or 3 Estrogen Receptor-Positive Breast Cancer.” Clin Cancer Res 23(15): 4055-4065.
8. Gao Q, Lopez-Knowles E, MC U C, et al: Major Impact of Sampling Methodology on Gene Expression in Estrogen Receptor-Positive Breast Cancer. JNCI Cancer Spectr 2:pky005, 2018.
9. Johnston et al., “Randomized Phase II Study Evaluating Palbociclib in Addition to Letrozole as Neoadjuvant Therapy in Estrogen Receptor-Positive Early Breast Cancer: PALLET Trial”, J Clin Oncol. 2019 Jan. 20; 37(3):178-189. doi: 10.1200/JCO.18.01624.

1. A method for predicting whether a human subject having breast cancer will be resistant to, or sensitive to, therapy with a cyclin-dependent kinase (CDK) inhibitor, the method comprising:
a) measuring the gene expression in a sample obtained from the the patient to obtain a sample gene expression profile of the breast tumour of at least the following modules:
(i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) an RB1 module comprising the gene RB1; and
(iv) a CCNE1 module comprising the gene CCNE1; and
b) making a prediction of whether the subject will be resistant to or sensitive to said CDK inhibitor treatment based on the sample gene expression profile comprising said modules (i) to (iv).

2. The method of claim 1, wherein the luminal vs. non-luminal module comprises the genes: ANLN, ESR1, PGR and SLC39A6.

3. The method of claim 2, wherein the luminal vs. non-luminal module comprises the genes: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T.

4. The method of claim 1, wherein the E2F module comprises the genes: SFRS1, DNAJC9, FBXO5, DCK, and TMPO.

5. The method of claim 4, wherein the E2F module comprises the genes: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SFRS10, SFRS7, SNRPD1, STMN1 and TMPO.

6. The method of claim 1, wherein the method further comprises measuring the gene expression in the sample of one or more housekeeping genes.

7. The method of claim 6, wherein the housekeeping genes comprise at least 2, 3, 4, 5, 6, 7, or at least 8 housekeeping genes selected from the group consisting of: ACTB, MRPL19, PSMC4, RPLP0, SF3A1, GUSB (alias GUS), PUM1 and TFRC.

8. The method of claim 1, wherein the subject is predicted to be resistant to said CDK inhibitor therapy when at least one of the following is true:
(i) the luminal vs. non-luminal module classifies the sample as non-luminal;
(ii) the E2F module classifies the sample as having high E2F expression;
(iii) the RB1 module classifies the sample as having low RB1 expression; and
(iv) the CCNE1 module classifies the sample as having high CCNE1 expression,
and wherein the subject is predicted to be sensitive to said CDK inhibitor therapy otherwise.

9. The method of claim 1, wherein:
(i) the luminal vs. non-luminal module classifies the sample as luminal;
(ii) the E2F module classifies the sample as having low E2F expression;
(iii) the RB1 module classifies the sample as having high RB1 expression; and
(iv) the CCNE1 module classifies the sample as having low CCNE1 expression,
and wherein the subject is predicted to be sensitive to said CDK inhibitor therapy.

10. The method of claim 8, wherein the E2F module classifies a sample as having high E2F expression when the average log₂ gene expression of E2F signature genes is greater than or equal to 9.392 or is greater than or equal to 9.4462.

11. The method of claim 8, wherein the RB1 module classifies the sample as having low RB1 gene expression when the log₂ gene expression measures less than or equal to 8.4068 or measures less than or equal to 8.4332.

12. The method of claim 8, wherein the CCN1E module classifies the sample as having high CCN1E expression when the log₂ gene expression measures greater than or equal to 8.264 or measures greater than or equal to 7.9596.

13. The method of claim 8, wherein the luminal vs. non-luminal module classifies the sample as luminal or non-luminal on the basis of the nearest centroid, wherein the sample gene expression profile of the genes of said luminal vs. non-luminal module is compared with reference centroids derived from measured gene expression of the said genes from a plurality of samples known to be of luminal phenotype and a plurality of samples known to be of non-luminal phenotype, respectively.

14. The method of claim 13, wherein the genes of the luminal vs. non-luminal module and corresponding reference centroids are selected from the following a) to f):

| Genes | Luminal | Non-Luminal |
|---|---|---|
| a) | | |
| ANLN | −0.436597966 | −0.171060579 |
| ESR1 | 0.724924673 | −0.293827849 |
| PGR | −0.14811616 | −0.58065462 |
| SLC39A6 | 2.236554031 | 1.642473045 |
| b) | | |
| ANLN | −0.436597966 | −0.171060579 |
| BCL2 | 0.214783191 | −0.257430908 |
| ESR1 | 0.724924673 | −0.293827849 |
| PGR | 0.243228161 | −0.14811616 |
| PHGDH | −0.008631878 | 0.414898718 |
| SLC39A6 | 2.236554031 | 1.642473045 |
| c) | | |
| ANLN | −0.436597966 | −0.171060579 |
| BCL2 | 0.214783191 | −0.257430908 |
| CENPF | 0.261621563 | 0.531497672 |
| ESR1 | 0.724924673 | −0.293827849 |
| PGR | 0.243228161 | −0.14811616 |
| PHGDH | −0.391826227 | −0.008631878 |
| RRM2 | 0.59104305 | 0.842673949 |
| SLC39A6 | 2.236554031 | 1.642473045 |
| d) | | |
| ANLN | −0.436597966 | −0.171060579 |
| BCL2 | 0.214783191 | −0.257430908 |
| CDC20 | −0.347286987 | −0.09561991 |
| CDH3 | −0.522006879 | −0.173025486 |
| CENPF | 0.261621563 | 0.531497672 |
| ESR1 | 0.724924673 | −0.293827849 |
| PGR | 0.243228161 | −0.14811616 |
| PHGDH | −0.391826227 | −0.008631878 |
| RRM2 | 0.363376998 | 0.59104305 |
| SLC39A6 | 2.236554031 | 1.642473045 |
| e) | | |
| ANLN | −0.436597966 | −0.171060579 |
| BCL2 | 0.214783191 | −0.257430908 |
| CDC20 | −0.347286987 | −0.09561991 |
| CDH3 | −0.522006879 | −0.173025486 |
| CENPF | 0.017447942 | 0.261621563 |
| ESR1 | 0.724924673 | −0.293827849 |
| PGR | 0.243228161 | −0.14811616 |
| PHGDH | −0.391826227 | −0.008631878 |
| PTTG1 | 0.028806637 | 0.23337021 |
| RRM2 | 0.363376998 | 0.59104305 |
| SLC39A6 | 2.236554031 | 1.642473045 |
| UBE2C | 0.060331049 | 0.318978765 |
| f) | | |
| ANLN | −0.436597966 | −0.171060579 |
| BCL2 | 0.214783191 | −0.257430908 |
| CDC20 | −0.574985771 | −0.347286987 |
| CDH3 | −0.522006879 | −0.173025486 |
| CENPF | 0.017447942 | 0.261621563 |
| ESR1 | 0.724924673 | −0.293827849 |
| FOXA1 | 0.774898643 | 0.173550394 |
| MLPH | 0.825757051 | 0.357073665 |
| PGR | 0.243228161 | −0.14811616 |
| PHGDH | −0.391826227 | −0.008631878 |
| PTTG1 | 0.028806637 | 0.23337021 |
| RRM2 | 0.363376998 | 0.59104305 |
| SLC39A6 | 2.236554031 | 1.642473045 |
| UBE2C | 0.060331049 | 0.318978765 |

15-25. (canceled)

26. The method of claim 1, wherein the subject is predicted to be sensitive to said CDK inhibitor therapy, and wherein the method further comprises the step of administering a therapeutically effective amount of a CDK inhibitor.

27-29. (canceled)

30. The method of claim 1, wherein the subject is predicted to be resistant to said CDK inhibitor therapy, and wherein the method further comprises administering endocrine therapy to the subject in the absence of any CDK4/6 inhibitor therapy.

31. A computer-implemented method for predicting whether a human subject having breast cancer will be resistant to, or sensitive to, therapy with a cyclin-dependent kinase (CDK) inhibitor, the method comprising:
a) obtaining gene expression data representing the gene expression profile of a sample obtained from the breast tumour of the subject of at least the following modules:
(i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) an RB1 module comprising the gene RB1; and
(iv) a CCNE1 module comprising the gene CCNE1; and
b) comparing the gene expression data obtained in a) with reference gene expression profiles for each of said modules, optionally wherein the gene expression data and the reference gene expression profiles comprise gene expression measurements that have been normalised to one or more housekeeping genes;
c) classifying the subject as resistant to said CDK inhibitor therapy if at least one of the following is true:
(i) the luminal vs. non-luminal module classifies the sample as non-luminal;
(ii) the E2F module classifies the sample as having high E2F expression;
(iii) the RB1 module classifies the sample as having low RB1 expression; and
(iv) the CCNE1 module classifies the sample as having high CCNE1 expression,
or classifying the subject as sensitive to said CDK inhibitor therapy otherwise.

32. (canceled)

33. A system for predicting treatment response of a human subject having breast cancer to therapy with a cyclin-dependent kinase (CDK) inhibitor, the system comprising:
A) a plurality of oligonucleotide probes for detection of gene transcripts of the following genes:
(i) at least four genes from the luminal vs. non-luminal module consisting of the genes: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) at least five genes from the E2F module consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) the RB1 gene; and
(iv) the CCNE1 gene;
B) a computer having at least one processor and at least one non-transitory computer readable medium containing instructions that, when executed by the at least one processor, cause the at least one processor to perform operations comprising:
(a) receiving gene expression data representing the gene expression profile of a sample obtained from the breast tumour of a human subject having breast cancer of at least the following modules:
(i) a luminal vs. non-luminal module comprising at least four genes selected from the group consisting of: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS, UBE2C and UBE2T;
(ii) a E2F module comprising at least five genes selected from the group consisting of: ARHGAP11A, ATAD2, C10ORF119, CASP8AP2, CLSPN, DCK, DNAJC9, FANCD2, FBXO5, FKBP5, H2AFZ, KIAA0101, KPNB1, NUP62, RANBP1, RET, SFRS1, SERS10, SFRS7, SNRPD1, STMN1 and TMPO;
(iii) an RB1 module comprising the gene RB1; and
(iv) a CCNE1 module comprising the gene CCNE1; and
(b) comparing the gene expression data with reference gene expression profiles for each of said modules, optionally wherein the gene expression data and the reference gene expression profiles comprise gene expression measurements that have been normalised to one or more housekeeping genes; and
(c) classifying the subject as resistant to said CDK inhibitor therapy if at least one of the following is true:
(i) the luminal vs. non-luminal module classifies the sample as non-luminal;
(ii) the E2F module classifies the sample as having high E2F expression;
(iii) the RB1 module classifies the sample as having low RB1 expression; and
(iv) the CCNE1 module classifies the sample as having high CCNE1 expression,
or classifying the subject as sensitive to said CDK inhibitor therapy otherwise.

34-39. (canceled)

40. The method of claim 26, wherein the CDK inhibitor administered to the subject is a CDK4/6 inhibitor.

41. The method of claim 40, wherein the CDK4/6 inhibitor administered to the subject is selected from: palbociclib, abemaciclib and ribociclib.
